(19) United States
(12) Patent Application Publication
(10) Pub. No.: US 2024/0057856 A1
(43) Pub. Date: Feb. 22, 2024

(54) SYSTEMS AND METHODS FOR CONTROLLING A SURGICAL PUMP USING ENDOSCOPIC VIDEO DATA

(71) Applicant: Stryker Corporation, Kalamazoo, MI (US)

(72) Inventors: Dana Emad Mohamed ABDULQADER, Folsom, CA (US); Brady Lewis WOOLFORD, Mapleton, UT (US)

(73) Assignee: Stryker Corporation, Kalamazoo, MI (US)

(21) Appl. No.: 18/452,483

(22) Filed: Aug. 18, 2023

Related U.S. Application Data
(60) Provisional application No. 63/373,042, filed on Aug. 19, 2022.

(51) Int. Cl. A61B 1/015; A61B 1/00; A61B 1/317

(52) U.S. Cl. CPC A61B 1/015; A61B 1/00009; A61B 1/00039; A61B 1/317

(57) ABSTRACT

A method for coordinating the operation of a surgical instrument and a surgical pump to control fluid pressure in an internal anatomy of a patient during a surgical procedure includes: receiving video data captured by an imaging device configured to image the internal anatomy of the patient; automatically determining a presence of a surgical instrument in the internal anatomy of the patient based on the received video data; and in response to determining that the surgical instrument is present in the internal anatomy of the patient, initiating a suction associated with the surgical instrument prior to activation of the surgical instrument.

### CROSS-REFERENCE TO RELATED APPLICATION

This application claims the benefit of U.S. Provisional Application No. 63/373,042, filed Aug. 19, 2022, the entire content of which is incorporated herein by reference.

### FIELD

This disclosure relates to controlling an arthroscopy fluid pump configured to irrigate an internal area of a patient during a minimally invasive surgical procedure, and more specifically, to using video data taken from an endoscopic imaging device to automatically control the amount and pressure of fluid pumped into the internal area of the patient.

### BACKGROUND

Minimally invasive surgery generally involves the use of a high-definition camera coupled to an endoscope inserted into a patient to provide a surgeon with a clear and precise view within the body. When the endoscope is inserted into the internal area of a patient's body prior to or during a minimally invasive surgery, it is important to maintain an environment within the internal area that is conducive to clearly visualizing the area by the camera. For instance, keeping the internal area clear of blood, debris, or other visual impairments are critical to ensuring that a surgeon or other practitioner has adequate visibility of the internal area.

One way to keep an internal area relatively free and clear of visual disturbances during an endoscopic procedure is to irrigate the internal area with a clear fluid such as saline during the procedure. Irrigation involves introducing a clear fluid into the internal area at a particular rate (i.e., inflow), and removing the fluid by suction (i.e., outflow) such that a desired fluid pressure is maintained in the internal area. The constant flow of fluid can serve two purposes. First, the constant flow of fluid through the internal area of the patient can help to remove debris from the field of view of the imaging device, as the fluid carries the debris away from the area and is subsequently suctioned out of the area. Second, the fluid creates a pressure build up in the internal area which works to suppress bleeding by placing pressure on blood vessels in or around the internal area.

Irrigating an internal area during a minimally invasive surgery comes with risks. Applying too much pressure to a joint or other internal area of a patient can cause injury to the patient and can even permanently damage the area. Thus, during an endoscopic procedure, the fluid delivered to an internal area is managed to ensure that the pressure is high enough to keep the internal area clear for visualization, but low enough so as to not cause the patient harm. Surgical pumps can be utilized to perform fluid management during an endoscopic procedure. Surgical pumps regulate the inflow and outflow of irrigation fluid to maintain a particular pressure inside an internal area being visualized. The surgical pump can be configured to allow for the amount of pressure to be applied to an internal area to be adjusted during a surgery.

The amount of pressure needed during a surgery can be dynamic depending on a variety of factors. For instance the amount of pressure to be delivered can be based on the joint being operated on, the amount of bleeding in the area, as well the absence or presence of other instruments. Having the surgeon manually manage fluid pressure during a surgery can place a substantial cognitive burden on them. The surgeon has to ensure that the pump is creating enough pressure to allow for visualization of the internal area, while simultaneously minimizing the pressure in the internal area so as to prevent injury or permanent damage to the patient. In an environment where the pressure needs are constantly changing based on conditions during the operation, the surgeon will have to constantly adjust the pressure settings of the pump to respond to the changing conditions. These constant adjustments can be distracting, and reduce the amount of attention that the surgeon has towards the actual procedure itself.

### SUMMARY

According to an aspect, video data taken from an endoscopic imaging device can be used to automatically control a surgical pump for purposes of regulating fluid pressure in an internal area of a patient during an endoscopic procedure. The endoscopic imaging device can be pre-inserted into the internal area of the patient. The video data can comprise images of the internal area of the patient. The video data can comprise a time sequence of images of the internal area of the patient. In one or more examples, control of the surgical pump can be based in part on one or more features extracted from video data received from an endoscopic imaging device. The features can be extracted from the video data using a one or more machine learning models configured to determine the presence of various features within the images of the internal area of the patient. Optionally, the one or more machine learning models can be configured to determine the presence of a surgical tool that has the capability to provide suction to the internal area, such as a cutter tool or RF probe that can provide suction. The surgical tool can be pre-inserted into the internal area of the patient. Based on the determination, a control command can be generated for controlling the surgical pump to transition the source of suction in the internal area from a dedicated suction instrument to the surgical tool determined to be present in the internal area. Based on the determination, the surgical pump can be controlled to transition the source of suction in the internal area from a dedicated suction instrument to the surgical tool determined to be present in the internal area.

According to an aspect, a method for coordinating the operation of a surgical instrument and a surgical suction pump, can comprise comprising receiving, by a processor, video data comprising images of an internal anatomy of a patient; the processor processing the received video data for automatically determining a presence of a surgical instrument in the images; and the processor, in response to determining that the surgical instrument is present in the images, generating an initiation command for initiating a suction associated with the surgical instrument prior to activation of the surgical instrument. According to an aspect, a method for coordinating the operation of a surgical instrument and a surgical pump to control fluid pressure in an internal anatomy of a patient during a surgical procedure includes: receiving video data captured by an imaging device configured to image the internal anatomy of the patient; automatically determining a presence of a surgical instrument in the internal anatomy of the patient based on the received video data; and in response to determining that the surgical instrument is present in the internal anatomy of the patient, initiating a suction associated with the surgical instrument prior to activation of the surgical instrument.

Optionally, the method includes generating an adjustment command for adjusting one or more characteristics of fluid inflow to the internal anatomy generated by the surgical pump. Optionally, the method includes adjusting one or more characteristics of fluid inflow to the internal anatomy generated by the surgical pump.

Optionally, the method includes the processor determining that the surgical instrument has been deactivated; in response to determining that the surgical tool has been deactivated: the processor determining whether the surgical instrument is still present in the images of the internal anatomy of the patient, and the processor, in response to determining that the surgical instrument is still present in the images, generating a command for maintaining at least some of the suction associated with the surgical instrument. Optionally, the method includes: determining that the surgical instrument has been deactivated; in response to determining that the surgical tool has been deactivated: determining whether the surgical instrument is still present in the internal anatomy of the patient based on the received video data, and in response to determining that the surgical instrument is still present in the internal anatomy of the patient, maintaining at least some of the suction associated with the surgical instrument.

Optionally, maintaining at least some of the suction comprises reducing an amount of the suction. For instance, the command for maintaining at least some of the suction comprises a command for reducing an amount of the suction.

Optionally, the method includes: the processor determining that the surgical instrument has been removed from the images of the internal anatomy of the patient; and the processor, in response to determining that the surgical instrument has been removed from the images, generating a deactivation command for deactivating the suction associated with the surgical instrument. The processor can e.g. be configured to determine whether, during a time sequence of images of the internal anatomy of the patient, the surgical instrument is absent in images of the sequence. The processor can e.g. be configured to determine whether, during a time sequence of images of the internal anatomy of the patient, the surgical instrument is present in first images of the sequence, and the surgical instrument is absent in second images of the sequence, the second images being associated with a later moment in time than the first images. Optionally, the method includes: determining that the surgical instrument has been removed from the internal anatomy of the patient based on the received video data; and in response to determining that the surgical instrument has been removed from the internal anatomy of the patient, deactivating the suction associated with the surgical instrument.

Optionally, automatically determining a presence of a surgical instrument in the images of the internal anatomy of the patient comprises using one or more classifiers to determine the presence of the surgical instrument in the internal anatomy of the patient. Optionally, automatically determining a presence of a surgical instrument in the internal anatomy of the patient based on the received video data comprises using one or more classifiers to determine the presence of the surgical instrument in the internal anatomy of the patient.

Optionally, the one or more classifiers are trained using one or more training images annotated with types of instruments pictured in the one or more training images.

Optionally, the one or more classifiers are generated using at least one of an unsupervised training process, inductive learning, deductive learning, or reinforcement learning.

Optionally, the one or more classifiers are configured to identify at least one of a radio frequency (RF) probe, a bur tool, and a cutter tool.

Optionally, the method includes the processor determining that an instrument supplying a fluid flow to the internal anatomy of the patient has been removed from the images of the internal anatomy of the patient by applying the one or more classifiers to the received video data; and the processor, in response to determining that the instrument supplying the fluid flow to the internal anatomy of the patient has been removed from the images, generating a flow decrease command for decreasing a flow through the surgical pump. Optionally, the method includes: determining that an instrument supplying a fluid flow to the internal anatomy of the patient has been removed from the internal anatomy of the patient by applying the one or more classifiers to the received video data; and in response to determining that the instrument supplying the fluid flow to the internal anatomy of the patient has been removed from the internal anatomy of the patient, decreasing a flow through the surgical pump.

Optionally, the method includes the processor determining that the internal anatomy of the patient is collapsing based on the images of the internal anatomy of the patient; and in response to determining that the internal anatomy of the patient is collapsing, generating a suction decrease command for decreasing the suction associated with the surgical instrument. Optionally, the method includes: determining that the internal anatomy of the patient is collapsing based on the received video data; and in response to determining that the internal anatomy of the patient is collapsing, decreasing the suction associated with the surgical instrument.

Optionally, the suction decrease command is generated in response to determining that the internal anatomy of the patient is collapsing only if the surgical instrument is active. Optionally, the suction associated with the surgical instrument is decreased in response to determining that the internal anatomy of the patient is collapsing only if the surgical instrument is active.

Optionally, the method includes generating an increase command for increasing at least one of fluid inflow pressure and fluid inflow flow rate in response to determining that the internal anatomy of the patient is collapsing. Optionally, the method includes increasing at least one of fluid inflow pressure and fluid inflow flow rate in response to determining that the internal anatomy of the patient is collapsing.

Optionally, the method includes: receiving a user selection to switch a mode of operation of the surgical pump; and in response to receiving the user selection, generating a mode switch command for switching the mode of operation by adjusting at least one of: (i) the suction associated with the surgical instrument and (ii) one or more characteristics of fluid inflow to the internal anatomy generated by the surgical pump. Optionally, the method includes: receiving a user selection to switch a mode of operation of the surgical pump; and in response to receiving the user selection, switching the mode of operation by adjusting at least one of: (i) the suction associated with the surgical instrument and (ii) one or more characteristics of fluid inflow to the internal anatomy generated by the surgical pump.

Optionally, the mode switch command is configured for switching to a visualization mode, and adjusting the at least one of the suction associated with the surgical instrument and the one or more characteristics of fluid inflow to the internal anatomy generated by the surgical pump to increase at least one of a pressure and a rate of flow of fluid in the internal anatomy of the patient. Optionally, switching the mode comprises switching to a visualization mode, and wherein the at least one of the suction associated with the surgical instrument and the one or more characteristics of fluid inflow to the internal anatomy generated by the surgical pump is adjusted to increase at least one of a pressure and a rate of flow of fluid in the internal anatomy of the patient.

Optionally, the mode switch command is configured for switching to a pressure reduction mode, and adjusting the at least one of the suction associated with the surgical instrument and the one or more characteristics of fluid inflow to the internal anatomy generated by the surgical pump to decrease the fluid pressure in the internal anatomy of the patient. Optionally, switching the mode comprises switching to a pressure reduction mode, and wherein the at least one of the suction associated with the surgical instrument and the one or more characteristics of fluid inflow to the internal anatomy generated by the surgical pump is adjusted to decrease the fluid pressure in the internal anatomy of the patient.

Optionally, the mode switch command is configured for switching from a visualization mode or a pressure reduction mode to a balanced mode, and adjusting the at least one of the suction associated with the surgical instrument and the one or more characteristics of fluid inflow to the internal anatomy to control the fluid pressure in the internal anatomy of the patient to be lower than a fluid pressure associated with the visualization mode and higher than a fluid pressure associated with the extravasation mode. Optionally, switching the mode comprises switching from a visualization mode or a pressure reduction mode to a balanced mode, and wherein the at least one of the suction associated with the surgical instrument and the one or more characteristics of fluid inflow to the internal anatomy is adjusted to control the fluid pressure in the internal anatomy of the patient to be lower than a fluid pressure associated with the visualization mode and higher than a fluid pressure associated with the extravasation mode.

Optionally, the method includes: receiving blood pressure information from a blood pressure monitoring device; and generating an adjustment command for adjusting at least one of: (i) the suction associated with the surgical instrument and (ii) one or more characteristics of fluid inflow to the internal anatomy generated by the surgical pump based on the received blood pressure information. Optionally, the method includes: receiving blood pressure information from a blood pressure monitoring device; and adjusting at least one of: (i) the suction associated with the surgical instrument and (ii) one or more characteristics of fluid inflow to the internal anatomy generated by the surgical pump based on the received blood pressure information. In some examples, the system can decrease the inflow parameters (e.g., pressure, inflow rate) if the joint pressure is higher than the blood pressure measured by the blood pressure monitor and/or if the difference between the joint pressure and the blood pressure exceeds a predefined threshold.

Optionally, the adjustment command is configured for adjusting the at least one of the suction associated with the surgical instrument and the one or more characteristics of fluid inflow to the internal anatomy generated by the surgical pump to increase a pressure in the internal anatomy above a mean arterial pressure. Optionally, the at least one of the suction associated with the surgical instrument and the one or more characteristics of fluid inflow to the internal anatomy generated by the surgical pump is adjusted to increase a pressure in the internal anatomy above a mean arterial pressure.

Optionally, the method includes: automatically determining a presence of blood in the images of the internal anatomy of the patient, and in response to determining the presence of the blood in the images, generating a command for adjusting the at least one of the suction associated with the surgical instrument and the one or more characteristics of fluid inflow to the internal anatomy generated by the surgical pump based on the received blood pressure information. Optionally, the method includes automatically determining a presence of blood in the internal anatomy of the patient based on the received video data, and in response to determining the presence of the blood in the internal anatomy of the patient, adjusting the at least one of the suction associated with the surgical instrument and the one or more characteristics of fluid inflow to the internal anatomy generated by the surgical pump based on the received blood pressure information.

Optionally, the method includes: determining that the suction associated with the surgical instrument has decreased; and in response to determining that the suction associated with the surgical instrument has decreased, generating a command for increasing suction provided by a different surgical instrument. Optionally, the method includes: determining that the suction associated with the surgical instrument has decreased; and in response to determining that the suction associated with the surgical instrument has decreased, increasing suction provided by a different surgical instrument.

Optionally, the different surgical instrument is a dedicated suction device of the surgical pump.

According to an aspect, a system includes one or more processors, and e.g. memory, and one or more programs stored in the memory, wherein the processors are configured, e.g. by means of the programs, for: receiving video data, such as captured by an imaging device, comprising images of an internal anatomy of a patient; automatically determining a presence of a surgical instrument in the images; and in response to determining that the surgical instrument is present in the images, generating an initiation command for initiating a suction associated with the surgical instrument prior to activation of the surgical instrument. According to an aspect, a system includes one or more processors, memory, and one or more programs stored in the memory for execution by the one or more processors and including instructions for: receiving video data captured by an imaging device configured to image the internal anatomy of the patient; automatically determining a presence of a surgical instrument in the internal anatomy of the patient based on the received video data; and in response to determining that the surgical instrument is present in the internal anatomy of the patient, initiating a suction associated with the surgical instrument prior to activation of the surgical instrument.

Optionally, the system includes an image processing system configured for receiving the video data captured by the imaging device and automatically determining the presence of the surgical instrument in the images. Optionally, the system includes a surgical pump configured for, in response to receiving the initiation command, initiating the suction associated with the surgical instrument prior to activation of the surgical instrument. Optionally, the system includes an image processing system that receives the video data captured by the imaging device and automatically determines the presence of the surgical instrument in the internal anatomy of the patient based on the received video data, and a surgical pump that initiates the suction associated with the surgical instrument prior to activation of the surgical instrument.

Optionally, the one or more processors are configured for generating an adjustment command for adjusting one or more characteristics of fluid inflow to the internal anatomy generated by the surgical pump. Optionally, the one or more programs include instructions for adjusting one or more characteristics of fluid inflow to the internal anatomy generated by the surgical pump.

Optionally, the one or more processors are configured for: determining that the surgical instrument has been deactivated; in response to determining that the surgical tool has been deactivated: determining whether the surgical instrument is still present in the images, and in response to determining that the surgical instrument is still present in the images, generating a command for maintaining at least some of the suction associated with the surgical instrument. The processor can e.g. be configured to determine whether, during a time sequence of images of the internal anatomy of the patient, a surgical instrument present in preceding images of the sequence is still present in a current image of the sequence. Optionally, the one or more programs include instructions for: determining that the surgical instrument has been deactivated; in response to determining that the surgical tool has been deactivated: determining whether the surgical instrument is still present in the internal anatomy of the patient based on the received video data, and in response to determining that the surgical instrument is still present in the internal anatomy of the patient, maintaining at least some of the suction associated with the surgical instrument.

Optionally, the command for maintaining at least some of the suction comprises a command for reducing an amount of the suction. Optionally, maintaining at least some of the suction comprises reducing an amount of the suction.

Optionally, the one or more processors are configured for: determining that the surgical instrument has been removed from images; and in response to determining that the surgical instrument has been removed from the images, generating a deactivation command for deactivating the suction associated with the surgical instrument. The processor can e.g. be configured to determine whether, during a time sequence of images of the internal anatomy of the patient, the surgical instrument is absent in images of the sequence. The processor can e.g. be configured to determine whether, during a time sequence of images of the internal anatomy of the patient, the surgical instrument is present in first images of the sequence, and the surgical instrument is removed from second images of the sequence, the second images being associated with a later moment in time than the first images. Optionally, the one or more programs include instructions for: determining that the surgical instrument has been removed from the internal anatomy of the patient based on the received video data; and in response to determining that the surgical instrument has been removed from the internal anatomy of the patient, deactivating the suction associated with the surgical instrument.

Optionally, the one or more processors are configured for automatically determining a presence of a surgical instrument in the images of the anatomy of the patient using one or more classifiers to determine the presence of the surgical instrument in the images. Optionally, wherein automatically determining a presence of a surgical instrument in the internal anatomy of the patient based on the received video data comprises using one or more classifiers to determine the presence of the surgical instrument in the internal anatomy of the patient.

Optionally, the one or more classifiers are trained using one or more training images annotated with types of instruments pictured in the one or more training images.

Optionally, the one or more classifiers are configured to identify at least one of a radio frequency (RF) probe, a bur tool, and a cutter tool.

Optionally, the one or more processors are configured for: determining that an instrument supplying a fluid flow to the internal anatomy of the patient has been removed from the images by applying the one or more classifiers to the received video data; and in response to determining that the instrument supplying the fluid flow to the internal anatomy of the patient has been removed from the images, generating a flow decrease command for decreasing a flow through the surgical pump. Optionally, the one or more programs include instructions for: determining that an instrument supplying a fluid flow to the internal anatomy of the patient has been removed from the internal anatomy of the patient by applying the one or more classifiers to the received video data; and in response to determining that the instrument supplying the fluid flow to the internal anatomy of the patient has been removed from the internal anatomy of the patient, decreasing a flow through the surgical pump.

Optionally, the one or more processors are configured for: determining that the internal anatomy of the patient is collapsing based on the images f the internal anatomy of the patient; and in response to determining that the internal anatomy of the patient is collapsing, generating a suction decrease command for decreasing the suction associated with the surgical instrument. Optionally, the one or more programs include instructions for: determining that the internal anatomy of the patient is collapsing based on the received video data; and in response to determining that the internal anatomy of the patient is collapsing, decreasing the suction associated with the surgical instrument.

Optionally, the suction decrease command is generated in response to determining that the internal anatomy of the patient is collapsing only if the surgical instrument is active. Optionally, the suction associated with the surgical instrument is decreased in response to determining that the internal anatomy of the patient is collapsing only if the surgical instrument is active.

Optionally, the one or more processors are configured for: receiving a user selection to switch a mode of operation of the surgical pump; and in response to receiving the user selection, generating a mode switch command for switching the mode of operation by adjusting at least one of: (i) the suction associated with the surgical instrument and (ii) one or more characteristics of fluid inflow to the internal anatomy generated by the surgical pump. Optinally, the one or more programs include instructions for: receiving a user selection to switch a mode of operation of the surgical pump; and in response to receiving the user selection, switching the mode of operation by adjusting at least one of: (i) the suction associated with the surgical instrument and (ii) one or more characteristics of fluid inflow to the internal anatomy generated by the surgical pump.

Optionally, the mode switch command is configured for switching to a visualization mode, and adjusting the at least one of the suction associated with the surgical instrument and the one or more characteristics of fluid inflow to the internal anatomy generated by the surgical pump to increase at least one of a pressure and a rate of flow of fluid in the internal anatomy of the patient. Optionally, switching the mode comprises switching to a visualization mode, and wherein the at least one of the suction associated with the surgical instrument and the one or more characteristics of fluid inflow to the internal anatomy generated by the surgical pump is adjusted to increase at least one of a pressure and a rate of flow of fluid in the internal anatomy of the patient.

Optionally, the mode switch command is configured for switching to a pressure reduction mode, and adjusting the at least one of the suction associated with the surgical instrument and the one or more characteristics of fluid inflow to the internal anatomy generated by the surgical pump to decrease the fluid pressure in the internal anatomy of the patient. Optionally, switching the mode comprises switching to a pressure reduction mode, and wherein the at least one of the suction associated with the surgical instrument and the one or more characteristics of fluid inflow to the internal anatomy generated by the surgical pump is adjusted to decrease the fluid pressure in the internal anatomy of the patient.

Optionally, the mode switch command is configured for switching from a visualization mode or a pressure reduction mode to a balanced mode, and adjusting the at least one of the suction associated with the surgical instrument and the one or more characteristics of fluid inflow to the internal anatomy to control the fluid pressure in the internal anatomy of the patient to be lower than a fluid pressure associated with the visualization mode and higher than a fluid pressure associated with the extravasation mode. Optionally, switching the mode comprises switching from a visualization mode or a pressure reduction mode to a balanced mode, and wherein the at least one of the suction associated with the surgical instrument and the one or more characteristics of fluid inflow to the internal anatomy is adjusted to control the fluid pressure in the internal anatomy of the patient to be lower than a fluid pressure associated with the visualization mode and higher than a fluid pressure associated with the extravasation mode.

Optionally, the one or more processor are configured for: receiving blood pressure information from a blood pressure monitoring device; and generating an adjustment command for adjusting at least one of: (i) the suction associated with the surgical instrument and (ii) one or more characteristics of fluid inflow to the internal anatomy generated by the surgical pump based on the received blood pressure information. Optionally, the one or more programs include instructions for: receiving blood pressure information from a blood pressure monitoring device; and adjusting at least one of: (i) the suction associated with the surgical instrument and (ii) one or more characteristics of fluid inflow to the internal anatomy generated by the surgical pump based on the received blood pressure information.

Optionally, the adjustment command is configured for adjusting the at least one of the suction associated with the surgical instrument and the one or more characteristics of fluid inflow to the internal anatomy generated by the surgical pump to increase a pressure in the internal anatomy above a mean arterial pressure. Optionally, the at least one of the suction associated with the surgical instrument and the one or more characteristics of fluid inflow to the internal anatomy generated by the surgical pump is adjusted to increase a pressure in the internal anatomy above a mean arterial pressure.

Optionally, the one or more processors are configured for: automatically determining a presence of blood in the images of the internal anatomy of the patient, and in response to determining the presence of the blood in the images, generating a command for adjusting the at least one of the suction associated with the surgical instrument and the one or more characteristics of fluid inflow to the internal anatomy generated by the surgical pump based on the received blood pressure information. Optionally, the one or more programs include instructions for: automatically determining a presence of blood in the internal anatomy of the patient based on the received video data, and in response to determining the presence of the blood in the internal anatomy of the patient, adjusting the at least one of the suction associated with the surgical instrument and the one or more characteristics of fluid inflow to the internal anatomy generated by the surgical pump based on the received blood pressure information.

Optionally, the one or more processors are configured for: determining that the suction associated with the surgical instrument has decreased; and in response to determining that the suction associated with the surgical instrument has decreased, generating a command for increasing suction provided by a different surgical instrument. Optionally, the one or more programs include instructions for: determining that the suction associated with the surgical instrument has decreased; and in response to determining that the suction associated with the surgical instrument has decreased, increasing suction provided by a different surgical instrument.

Optionally, the different surgical instrument is a dedicated suction device of the surgical pump.

According to an aspect, a computer program product comprises one or more programs for execution by one or more processors of a computing system for causing the computing system to perform any of the above methods or combination of the above methods. According to an aspect, a non-transitory computer readable storage medium stores one or more programs for execution by one or more processors of a computing system for causing the computing system to perform any of the above methods or combination of the above methods.

It will be appreciated that any of the variations, aspects, features and options described in view of the systems apply equally to the methods and vice versa. It will also be clear that any one or more of the above variations, aspects, features and options can be combined.

### BRIEF DESCRIPTION OF THE FIGURES

The invention will now be described, by way of example only, with reference to the accompanying drawings, in which:

FIG. 1 illustrates an exemplary endoscopy system according to examples of the disclosure.

FIG. 2 illustrates an exemplary method for controlling a surgical pump according to examples of the disclosure.

FIG. 3 illustrates an exemplary image processing process flow according to examples of the disclosure.

FIG. 4 illustrates an exemplary method for annotating images according to examples of the disclosure.

FIG. 5 illustrates an exemplary instrument suction activation process according to examples of the disclosure.

FIG. 6 illustrates an exemplary inflow deactivation process according to examples of the disclosure.

FIG. 7 illustrates an exemplary image clarity based process for controlling a surgical pump according to examples of the disclosure.

FIG. 8 illustrates an exemplary process for detecting blood in an image according to examples of the disclosure.

FIG. 9 illustrates an exemplary endoscopic image with segmented bleed regions according to examples of the disclosure.

FIG. 10 illustrates an exemplary process for detecting debris in an image according to examples of the disclosure.

FIG. 11 illustrates an exemplary endoscopic image with identified debris clusters according to examples of the disclosure.

FIG. 12 illustrates an exemplary process for detecting a snow globe effect in an image according to examples of the disclosure.

FIG. 13 illustrates an exemplary endoscopic image with segmented snowy area regions according to examples of the disclosure.

FIG. 14 illustrates an exemplary process for detecting turbidity in an image according to examples of the disclosure.

FIG. 15 illustrates an exemplary process for adjusting the settings of a surgical pump based on the image clarity according to examples of the disclosure.

FIG. 16 illustrates an exemplary process for adjusting the settings of a surgical pump based on a selection of a mode of operation of the surgical pump.

FIG. 17 illustrates an exemplary computing system, according to examples of the disclosure.

### DETAILED DESCRIPTION

Reference will now be made in detail to implementations and examples of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Described herein are systems and methods for automatically controlling of a surgical pump using video data taken from an endoscopic imaging device. The surgical pump can be controlled for purposes of regulating fluid pressure and/or fluid flow rate in an internal area of a patient using video data taken from an endoscopic imaging device. The endoscopic imaging device may have been pre-inserted into the internal area prior to the start of the method. According to various examples, one or more image frames are extracted from a video feed recorded from the endoscopic imaging device a surgical procedure. The extracted image frames, in one or more examples, can be processed using one or more machine learning models that are configured to determine the existence of various features in the one or more image frames of the visualized internal area of a patient. For instance in one or more examples, the one or more machine learning models can be configured to determine the joint type depicted in the image, one or more instruments present in the image frame(s), and/or the presence/absence of visual disturbances present in the image frame(s).

According to an aspect, the features determined by the one or more machine learning models to be present in one or more image frames can be used to generate one or more control commands for controlling a surgical pump, such as control commands for adjusting a flow fluid setting of the surgical pump. The features determined by the one or more machine learning models to be present in one or more image frames can be used to determine an adjusted flow fluid setting of a surgical pump. The features determined by the one or more machine learning models to be present in one or more image frames can be used to control the fluid inflow to the internal area and/or the fluid outflow from the internal area provided by the surgical pump, such as to control a flow through and/or pressure in the internal area, or at least generate control commands thereto. In one or more examples, one or more aspects of the fluid inflow to and/or fluid outflow from the internal area provided by the surgical pump can be set based on what instruments are determined to be present in the internal area as depicted in the image frames extracted from the endoscopic video data. In one or more examples, the image frames can be processed by one or more classifiers of one or more machine learning models to determine whether an instrument is found in the image frames. If an instrument is detected, further classifiers can be applied to the image frames to determine if the surgical instrument is of the type that has its own suction (such as an RF probe, a cutter, grasper, anchor, drill, drill guide, reamer, suture, or a bur). In one or more examples, control commands can be generated and/or the surgical pump can be made to work with the suction capabilities of the surgical instruments found in an image so as to provide overall pressure management in the internal area. In one or more examples, suction provided by a surgical tool determined to be present in the internal area can be increased while suction provided by a dedicated suction instrument is decreased to control one or more aspects of the fluid flow in the internal area while suction is handed off from the dedicated suction instrument to the surgical tool. In one or more examples, this suction hand-off can be done prior to activation of the surgical tool so that when the user activates the surgical tool, some or all of the suction is provided by the surgical tool, which avoids an abrupt change in pressure that would occur if suction were switched upon activation of the surgical tool.

According to an aspect, control commands can be generated and/or a surgical pump can be controlled to adjust one or more characteristics of fluid flow in an internal area based on the determined presence or absence of visual disturbances detected in one or more image frames. Examples of visual disturbances that may be detected in one or more image frames are blood, bubbles, debris, snow globe effect, turbidity, etc. Based on the determined presence of these visual disturbances in one or more image frames, the surgical pump can be controlled to increase pressure, flow rate to the internal area, and/or flow rate from the internal area when these disturbances are detected and/or decrease pressure, flow rate to the internal area, and/or flow rate from the internal area when the disturbances are found to not be present.

In the following description of the various examples, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure in some examples also relates to a device for performing the operations described herein. This device may be specially constructed for the required purposes, or it may comprise a general purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each connected to a computer system bus. Furthermore, the computing systems referred to in the specification may include a single processor or may be architectures employing multiple processor designs, such as for performing different functions or for increased computing capability. Suitable processors include central processing units (CPUs), graphical processing units (GPUs), field programmable gate arrays (FPGAs), and ASICs. In one or more examples, the systems and methods presented herein, including the computing systems referred to in the specification may be implemented on a cloud computing and cloud storage platform.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present disclosure as described herein.

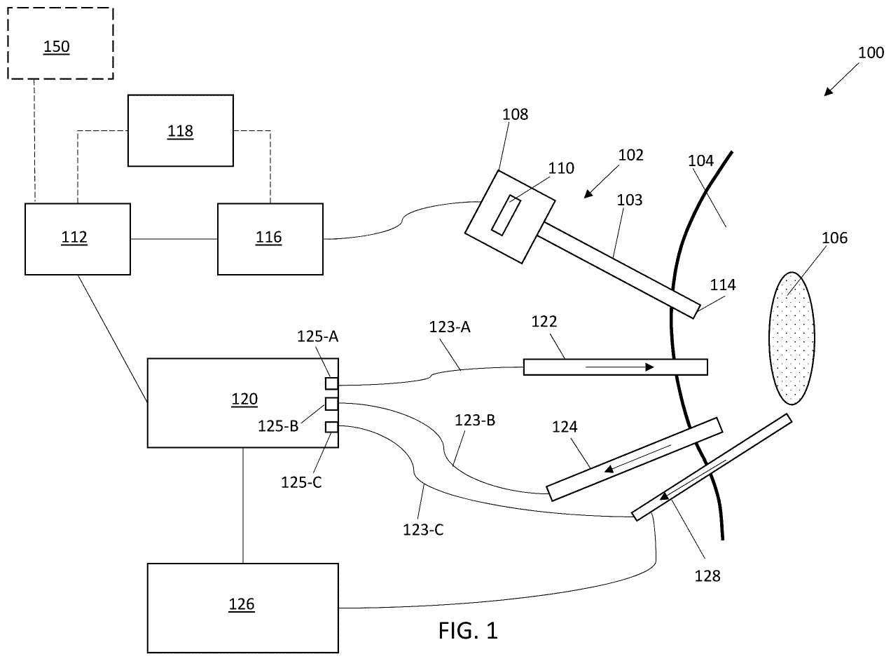

FIG. 1 illustrates an exemplary endoscopy system according to examples of the disclosure. System 100 includes an endoscopic imaging device 102 for insertion into a surgical cavity 104 for imaging tissue 106 within the surgical cavity 104 during a medical procedure. The endoscopic imaging device 102 may be pre-inserted into the surgical cavity 104 prior to performing the methods as described herein. The endoscopic imaging device 102 may include an endoscope 103 that extends from an endoscopic camera head 108 that includes one or more imaging sensors 110. Light reflected and/or emitted (such as fluorescence light emitted by fluorescing targets that are excited by fluorescence excitation illumination light) from the tissue 106 is received by the distal end 114 of the endoscope 103. The light is propagated by the endoscope 103, such as via one or more optical components (for example, one or more lenses, prisms, light pipes, or other optical components), to the camera head 108, where it is directed onto the one or more imaging sensors 110. In one or more examples, one or more filters (not shown) may be included in the endoscope 103 and/or camera head 108 for filtering a portion of the light received from the tissue 106 (such as fluorescence excitation light). While the example above describes an example implementation of an endoscopic imaging device, the example should not be seen as limiting to the disclosure and the systems and methods described herein can be implemented using other imaging devices that are configured to image the internal area of a patient.

The one or more imaging sensors 110 generate pixel data that can be transmitted to a camera control unit 116 that is communicatively connected to the camera head 108. The camera control unit 116 generates a video feed from the pixel data that shows the tissue being viewed by the endoscopic imaging device 102 at any given moment in time. In one or more examples, the video feed can be transmitted to an image processing unit 112 for further image processing, storage, display, and/or routing to one or more remote computing systems 150 such as a cloud computing system. The video feed or portions thereof can be transmitted to one or more displays 118, from the camera control unit 116 and/or the image processing unit 112, for visualization by medical personnel, such as by a surgeon for visualizing the surgical cavity 104 during a surgical procedure on a patient.

The image processing unit 112 can be communicatively coupled to an endoscopic surgical pump 120 configured to control the inflow and outflow of fluid in an internal portion of a patient. As described in further detail below, the image processing unit 112 can use the video data it processes to determine an adjusted pressure setting for the surgical pump 120, usable for regulating the pressure at an internal area of a patient such as surgical cavity 104. The image processing unit 112 can use the video data it processes to control the surgical pump 120 so as to regulate one or more characteristics (e.g., pressure and/or flow rate) of flow in an internal area of a patient such as surgical cavity 104. The surgical pump 120 can include an inflow instrument 122 configured to deliver a clear fluid such as saline into the surgical cavity 104 via a fluid supply line 123-A. The surgical pump 120 can also include a dedicated suction instrument 124 configured to suction fluid out of the surgical cavity 104 via a suction line 123-B. The inflow instrument 122 and the dedicated suction instrument 124 can be pre-inserted into the surgical cavity 104 prior to performing the methods as described herein. In one or more examples, the surgical pump 120 is configured to regulate the internal pressure of the surgical cavity by either increasing or decreasing the rate at which the inflow instrument 122 pumps fluid into the surgical cavity 104 and/or by increasing/decreasing the amount of suction at suction instrument 124. This can be done, for example, via an inflow control system 125-A (for example, a dedicated pump and/or valve system) for the inflow instrument 122 and/or dedicated suction control system 125-B (that includes, for example, a dedicated pump and/or valve system) for the suction instrument 124. In one or more examples, the surgical pump can also include a pressure sensor that is configured to sense the pressure inside of surgical cavity 104 during a surgical procedure.

The system 100 can also include a tool controller 126 that is configured to control and/or operate a tool 128 used in performing a minimally invasive surgical procedure in the surgical cavity 104. The tool 128 can be pre-inserted into the surgical cavity 104 prior to performing the methods as described herein. The tool controller (or even the tool itself) can be communicatively coupled to the surgical pump 120. As will be described in further detail below, the tool 128 may be configured for applying suction inside the surgical cavity 104 that can work to suction out fluids and debris from the surgical cavity 104. The suction applied by the tool 128 may be provided by the surgical pump 120 via suction line 123-C. The surgical pump 120 may be configured to control suction provided to the tool 128 using an instrument suction control system 125-C(that includes, for example, a dedicated pump and/or valve system). By communicatively coupling the tool 128 and the surgical pump 120, the surgical pump 120 can coordinate the actions of its own dedicated suction instrument 124 as well as suction provided to the tool 128 to regulate the pressure of the surgical cavity 104 as will be further described below. The suction provided by tool 128 can be provided by a suction unit that is part of the same device that provides suction to the suction instrument 124 and/or that provides fluid inflow the inflow instrument 122. In one or more examples, one or more separate devices can provide the suction to the tool 128, the suction to the suction instrument 124, and/or the fluid inflow the inflow instrument 122, where the one or more separate devices are communicatively coupled to a central controller that coordinate the separate devices. As used herein, the term surgical pump (e.g., surgical pump 120) encompasses a single device (e.g., a single off-the-shelf device) that provides the fluid inflow and suction and multiple communicatively interconnected devices that collectively provide controlled fluid inflow and suction. As used herein, the terms instrument and surgical instrument encompasses inflow instruments that provide fluid to the surgical cavity, dedicated suction instruments, and surgical tools.

As described above, different scenarios and conditions taking place inside of surgical cavity 104 can require that the inflow and/or outflow of surgical pump 120 be adjusted. For instance, the introduction into the surgical cavity, and/or operation, of a surgical tool capable of providing suction may require that suction handled by a dedicated suction instrument be at least partially handed off to the surgical tool. Furthermore, visibility conditions within a surgical cavity may require an increase or decrease in the inflow and/or outflow of surgical pump 120. For instance, an increase of blood within the surgical cavity 104 can require that the rate of inflow (which subsequently increases the pressure in the surgical cavity 104) be increased so as to arrest or minimize the bleeding. Conventionally, a surgeon would need to recognize a need to increase or decrease the pressure and then manually adjust the setting on the surgical pump to obtain the desired pressure. This process can interrupt the surgical procedure itself as the surgeon would need to stop with the procedure to make the necessary adjustments to the surgical pump 120, and further requires that the surgeon constantly assess whether the current pressure and/or flow rate in the surgical cavity 104 is correct for the given conditions of the surgery.

Automating the process of detecting features in one or more image frames that indicate conditions associated with changing surgical pump inflow and/or outflow, as well as the process of adjusting the inflow and/or outflow of the surgical pump, can thus reduce the cognitive load of the surgeon performing a surgery, but in one or more examples can also ensure that the pressure inside a surgical cavity is controlled with precision. In this way, the surgical pump can provide a sufficient amount of pressure and/or flow rate needed to manage the surgical cavity (i.e., provide good visualization), while at the same time ensuring that the pressure isn't so great as to cause injury or damage to the patient (i.e., by causing minimal extravasation). For example, a surgical pump that has a pressure range of up to 150 mmHg may be controlled such that the pressure is in the range of 15-75 mmHg.

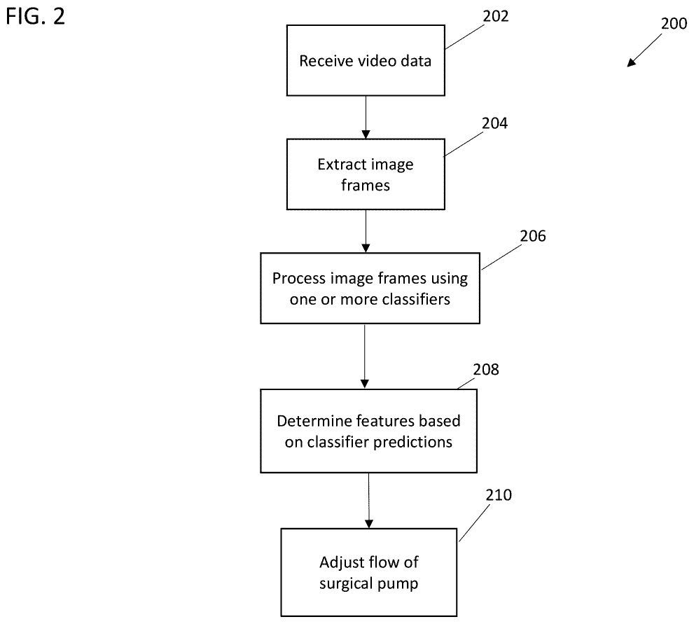

FIG. 2 illustrates an exemplary process 200 for controlling a surgical pump according to examples of the disclosure. Process 200 can be performed, for example, by one or more components of system 100 of FIG. 1. At step 202, video data from an endoscopic imaging device, such as endoscopic imaging device 102 of FIG. 1, or other type of imaging device is received at one or more processors configured to implement process 200, such as one or more processors of image processing unit 112 of FIG. 1. In one or more examples, the video data can be transmitted (e.g., by the imaging device 102 or by the camera control unit 116) to the one or more processors using a High-Definition Multimedia Interface (HDMI), Digital Visual Interface (DVI) or other interface capable of connecting a video source (such as an endoscopic camera) to a display device or graphics processor.

At step 204, one or more image frames can be extracted from the video data. In one or more examples, the one or more image frames can be extracted from the video data in a periodic interval at a pre-determined period. Alternatively or additionally, one or more image frames can be extracted from the video data in response to user input such as for instance the surgeon pushing a button or other user input device to indicate that they want to capture an image from the video data at any particular moment in time. In one or more examples, extracted image frames can be stored in memory according to known image storage standards for memory such as JPEG, GIF, PNG, and TIFF image file formats. In one or more examples, the pre-determined time between extracted image frames from the video data can be configured to ensure that an image is extracted during each stage in surgical procedure, thereby ensuring that the extracted images will adequately represent all of the steps in a surgical process. In one or more examples, the image frames can be extracted from the video data in real-time, i.e., as the surgical process is being performed. In one or more examples, and as part of step 204, the extracted image frames can be reduced in size and/or cropped so as to reduce the amount of memory required to store an image frame. In one or more examples, the process of extracting image frames from the received video data can be optional and the process 200 of FIG. 2 can be directly executed upon the video data from the endoscopic imaging device itself without requiring the extraction of image frames from the video feed.

At step 206, the one or more image frames are processed using one or more machine learning models that are configured to determine whether one or more extracted image frames include one or more features of interest. Step 206 can be performed, for example, by image processing unit 112 and/or by remote computing system 150.

In one or more examples, the one or more machine learning models can be configured to identify anatomy that is being shown in a given image. For instance, and as discussed in further detail below, the one or more machine learning models can be configured to identify a particular joint type shown in an image such as whether a given image is of a hip, a shoulder, a knee, or any other anatomical feature that can be viewed using an imaging device such as an endoscopic imaging device. Alternatively or additionally, the one or more machine learning models can be configured to determine what surgical instruments are present in one or more image frames. For example, the one or more machine learning models can be configured to determine that one or more of the inflow instrument 122, the dedicated suction instrument 124, and/or the tool 128 of FIG. 1 are present in one or more image frames. In one or more examples, the one or more machine learning models can be configured to determine the presence of one or more features that are associated with how clear or obscure a particular image is. For example, one or more machine learning models can be configured to determine the presence of blood, turbidity, bubbles, smoke, and/or other debris in a given image frame, which may indicate a need to increase the inflow of fluid from the surgical pump so as to remove the visual impairments from the surgical cavity. In one or more examples, the one or more machine learning models can be configured to determine whether a joint is collapsed and/or whether the distal end of the endoscopic imaging device is outside of the joint.

The one or more machine learning models can include one or more classifiers configured to determine what features are present in a given image frame or set of image frames. In one or more examples, the one or more machine learning models include a single classifier configured to determine the presence of multiple features in a given image frame or set of images frames. In one or more examples, the one or more machine learning models include one or more classifiers that determine the presence of just one feature. In one or more examples, an image frame can be analyzed by each of multiple classifiers. Alternatively, the processing of an image frame by a given classifier can depend upon the results of another classifier. As an example, a first classifier can be used to determine if a particular anatomical feature is present in a given image frame, and if the output of the classifier corresponds with it being more likely than not that the image contains a particular anatomical feature, then the image frame can be processed by a second classifier to determine what procedure step is shown in the image. For instance if it is determined that a particular image frame shows a hip joint, then that same image frame can also be processed by a classifier configured to determine if the image shows a torn labrum as well as a separate classifier configured to determine if the image shows a labrum post-repair. However, if the classifier configured to determine if a given image frame shows a hip joint determines that it is unlikely that the image frame shows a hip joint, then the image frame may not be processed by a classifier configured for determining a procedure step for a surgery involving a hip (i.e., a torn labrum or a repaired labrum).

The one or more classifiers are configured to generate a prediction that is indicative of whether or not a particular feature (that the classifier is configured to determine) exists within a particular image. Thus, rather than making a binary determination (yes or no) as to whether a particular image frame includes a particular feature, the prediction can inform the process as to how likely it is that a particular image frame includes a particular feature. As an example, a classifier that is configured to classify whether an image frame contains a hip joint can output a prediction in the range of 0 to 1 with 0 indicating that it is extremely unlikely that a particular image shows a hip joint and 1 indicating that it is extremely likely that a particular image frame shows a hip joint. Intermediate values between 0 and 1 can indicate the likelihood that an image frame contains a particular feature. For instance if a classifier outputs a 0.8, it can mean that it is more likely than not that the image frame shows a hip joint, while a prediction of 0.1 means that it is not likely that the image frame contains a hip joint.

The one or more classifiers can be implemented using one or more convolutional neural networks (CNNs). A CNN can include one or more layers, with each layer of the CNN configured to aide in the process of determining whether a particular image includes a feature. Alternatively or additionally, the CNNs can be configured as Region-based Convolutional Neural Networks (R-CNNs) that can not only determine if a particular image frame or set of image frames contains a feature, but also can identify the specific location in the image frame or set of image frames where the feature is shown. One or more classifiers can be trained using a supervised training process, an unsupervised training process, a self-supervised training process, a weakly-supervised training process, or a semi-supervised training process. One or more classifiers can be trained using inductive learning, deductive learning, or reinforcement learning. Different classifiers can be generated using different learning processes. Additionally or alternatively, one or more classifiers can be implemented using one or more Visual Transformers. In one or more examples, one or more classifiers can be trained to determine the presence of one or more features based on a series of image frames, such as to determine whether an event took place, is taking place, and/or is likely to take place. For example, a series of image frames can be processed by one or more classifiers to determine that a joint is about to collapse based on changes in joint appearance over time.

At step 208, a determination is made as to what features are present within a particular image. The determination made at step 208 can be based on the predictions output from each of the classifiers. As an example, each of the predictions generated by each of the classifiers can be compared to one or more pre-determined thresholds and if the predictions exceeds the pre-determined threshold, a determination is made that the image contains the feature corresponding to that classifier. As an example, if a classifier that processed an image frame outputs a prediction of 0.7 for the image frame, and the pre-determined threshold is set at 0.5, then at step 208, a determination is made that the image frame shows the feature associated with the classifier.

At step 210, an adjusted flow setting for flow through the surgical pump is determined based on the determined presence of one or more features in one or more image frames. A control command may be generated representative of the determined adjusted flow setting for flow through the surgical pump. With reference to FIG. 1, step 210 may be performed, for example, by surgical pump 120 based on information received from image processing unit 112 indicating the features determined by the image processing unit 112 to be present in one or more image frames. For example, surgical pump 120 may receive a notification from the image processing unit 112 that the image processing unit 112 determined that surgical tool 128 is present, e.g. has been inserted, in the surgical cavity 104, and in response, the surgical pump 120 may determine that suction for the surgical tool 128 should be initiated. Step 210 can include generating a control command for and/or adjusting the flow through the surgical pump based on the determined presence of one or more features in the one or more image frames. For example, surgical pump 120 may generate a control command for initiating, and/or initiate, suction for the surgical tool 128 after determining that the suction for the surgical tool 128 should be initiated based on receiving a notification that the surgical tool 128 is present in the surgical cavity. Adjusting the flow can include decreasing or increasing the flow rate of fluid pumped into a surgical cavity by the surgical pump, adjusting the overall pressure provided by the surgical pump to the surgical cavity, adjusting an amount of suction from the surgical cavity provided by the surgical pump, and/or adjusting which instrument(s) provide flow into the surgical cavity and/or suction from the surgical cavity. Dedicated control commands may be generated thereto. In one or more examples of the disclosure, the surgical pump can include one or more peristaltic pumps that control the joint pressure through increasing and decreasing the inflow rate and/or one or more valves that reduce and/or increase flow rate. Alternatively or additionally, the surgical pump can include one or more propellers that generate a head pressure, which can be used to drive the pressure in a joint or surgical cavity. Thus, in one or more examples, adjusting the surgical pump at step 210 can include adjusting both a flow driven pump and a pressure driven pump as described above. In one or more examples, the determination of an adjusted flow setting is not performed by the surgical pump, but rather, is performed by another computing system, which then instructs the surgical pump to adjust its flow setting accordingly. A control command including data representative of the adjusted flow setting may be transmitted by the other computing system to the surgical pump. For example, an image processing unit, such as imaging processing unit 112 of FIG. 1, may determine an adjusted flow setting for a surgical pump, such as surgical pump 120, and may instruct the surgical pump accordingly, and the surgical pump may respond to the instruction by executing the flow setting adjustment.

In one or more examples, process 200 can include taking actions other than generating a control command for adjusting, and/or adjusting, the surgical pump flow settings in response to determining features present in one or more image frames. For example, where features affecting clarity, such as blood, turbidity, bubbles, smoke, and/or other debris, are determined to be present in a one or more image frames, one or more machine learning models (e.g., a conditional GAN or a variational auto-encoder) may be used to improve clarity of the one or more image frames. This may be done in addition to adjusting surgical pump flow settings or may be done as an alternative to adjusting surgical pump flow settings.

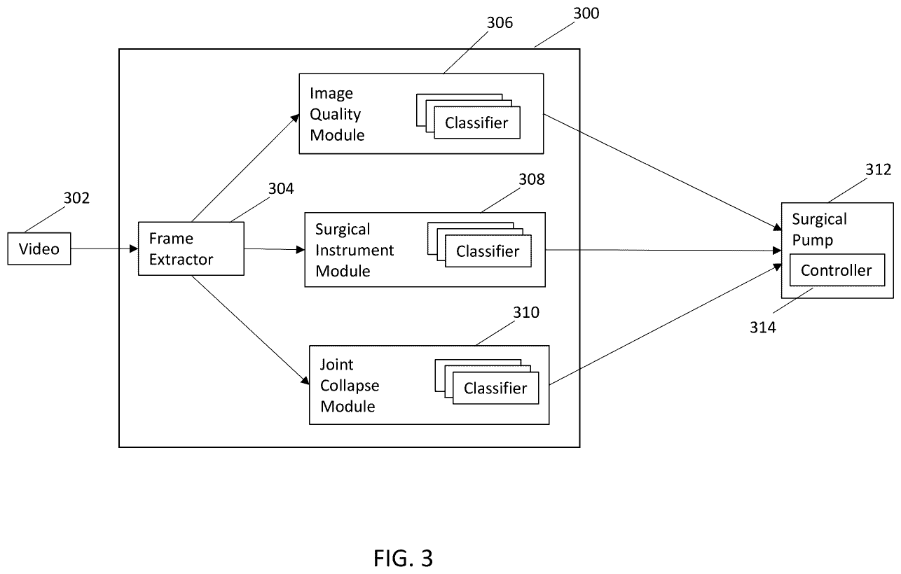

FIG. 3 is a functional block diagram of an exemplary image processing system according to examples of the disclosure. In one or more examples, the image processing system 300 can perform one or more steps of process 200 of FIG. 2, such as steps 202, 204, 206, and 208. As discussed further below, image processing system 300 includes one or more processors for extracting image frames from video and analyzing the image frames using one or more machine learning models to determine the presence of features in the image frames that correspond with conditions in a surgical cavity for which adjustments to fluid flow provided by a surgical pump 312 may be desired. The image processing system 300 can include a single processor or multiple processors. The image processing system 300 can be a single computing system, such as image processing unit 112 of FIG. 1, or can include multiple communicatively coupled computing systems, such as a combination of image processing unit 112 and remote computing system 150. Different functions of image processing system 300 can be implemented by different computing systems that are communicatively coupled to one another. For example, image frame extraction from video can be implemented by a local computing system that is located in an operating room and one or more machine learning models can be implemented by one or more remote computing systems, such as a cloud computing system, that receive the image frames from the local computing system and return information regarding detected features to the local computing system. The one or more processors of image processing system 300 can include one or more CPUs, one or more GPUs, one or more TPUs, and/or one or more FPGAs.

In one or more examples, the image processing system 300 receives video data 302, such as described above at step 202 of FIG. 2. In one or more examples, the image processing system 300 implements a frame extractor 304 for extracting one or more image frames from the video data 302, such as described above with respect to step 204 of FIG. 2. The image processing system 300 may implement one or more machine learning models for determining what features (if any) are present in a given image frame or set of image frames that may be associated with one or more conditions requiring adjustment to the flow settings and/or pressure settings of the surgical pump. The image processing system 300 may implement multiple modules, each having its own machine learning model(s) for determining the presence of different types of features. In the illustrated example, the image processing system 300 may include an image quality module 306 that may implement one or more machine learning models for determining whether features associated with poor image quality are present in the image frames, a surgical instrument module 308 that may implement one or more machine learning models for determining whether one or more surgical instruments are present in the image frames, and/or a joint collapse module 310 that may implement one or more machine learning models for determining whether or not a joint is collapsed. The machine learning models of each module can each include one or more classifiers for predicting the presence of features in the one or more image frames. In one or more examples, each module may make determinations regarding the presence or absence of features in the one or more images frames by comparing the predictions generated by the one or more classifiers to predetermined thresholds. For example, a module may determine that a given feature is present in one or more machine learning models when a prediction for the feature generated by a classifier is above a predetermined threshold. In one or more examples, classifier predictions are provided as outputs to an external system for the external system to make determinations regarding the presence or absence of features in the one or more images frames by comparing the predictions generated by the one or more classifiers to predetermined thresholds. As used herein, the term “module” refers to a software module and is not intended to refer to particular hardware. It will be understood by a person of ordinary skill that different modules can be implemented on the same computing hardware, on different computing hardware, or a combination thereof.

The surgical instrument module 308 can be configured to determine the presence of one or more surgical instruments in a given image frame. As will be described in detail further below, certain surgical instruments can include their own inflow or suction capabilities, which can influence the inflow and outflow rates of a surgical pump. Thus, in one or more examples, surgical instrument module 308 can include one or more classifiers configured to determine the presence (or absence) of various instruments in the surgical cavity that have inflow and/or suction capabilities, which can include determining a type of an instrument in the surgical cavity. For example, the one or more classifiers can be configured to determine the presence or absence of a cutter, a bur, grasper, anchor, drill, drill guide, reamer, suture, and/or an RF probe that has suction, or any other surgical instrument that has suction. In one or more examples, the surgical instrument module 308 can include multiple classifiers, each classifier configured to determine the presence of one or more types of surgical instruments, or can include a single classifier that can determine the presence of multiple types of surgical instruments. For instance, surgical instrument module 308 can include a first classifier configured to determine if a cutter is present in the surgical cavity and a second classifier configured to determine if a bur is present in the surgical cavity, or the surgical instrument module 308 can include a single classifier that can determine if a cutter and a bur is present in the surgical cavity. In one or more examples, surgical instrument module 308 can include one or more classifiers for determining that one or more fluid inflow instruments are present in the surgical cavity and/or that one or more suction instruments are present in the surgical cavity. In one or more examples, the one or more classifiers can determine that a dedicated suction instrument (e.g., suction instrument 124 of FIG. 1) is present in the surgical cavity and/or that a surgical tool that has suction capability (e.g., tool 128 of FIG. 1) is present in the surgical cavity.

In one or more examples, the surgical instrument module 308 can be configured to determine the presence of an instrument or portion of an instrument that is outside of the surgical cavity. For example, the surgical instrument module 308 can be configured to determine the presence of a shaver in one or more image frames. The presence of the shaver (to which a rotating tool, such as a bur or cutter, is connected) in the one or more image frames may indicate that the endoscopic imaging device has been removed from the surgical cavity. As such, a determination by the surgical instrument module 308 that a shaver is in the one or more image frames may be a trigger used (e.g., by a controller of the surgical pump) to reduce or shut off suction to the shaver and/or used as a trigger to reduce or shut off fluid inflow provided by the surgical pump. In one or more examples, the surgical instrument module 308 can be configured to determine a type of shaver (or other surgical instrument) located outside of the surgical cavity. Such a determination may be used to enable or disable functionality of the fluid pump based on a capability of the identified shaver type (or other surgical instrument type).

Image quality module 306 can be configured to determine the presence of various features associated with the clarity of one or more image frames. As described above, and described in detail below, various conditions that can inhibit the clarity of a video, such as blood, bubbles, debris, snow globe conditions, and turbidity can be mitigated by changing one or more settings of the surgical pump output, such as the pressure, inflow flow rate, and/or suction flow rate. Thus, in one or more examples, image quality module 306 includes one or more classifiers configured to determine the presence in one or more image frames of one or more of blood, bubbles, debris, snow globe conditions, and/or turbidity. Each feature relating to clarity can be determined by its own classifier or multiple features relating to clarity can be determined by a single classifier. In one or more examples, one or more classifiers can be configured to determine a degree of presence of a given feature. For example, a classifier for determining the presence of blood may determine three classes associated with blood—no blood, a relatively small amount of blood, and a relatively large amount of blood. These classes are merely exemplary, and it will be understood by one of skill in the art that any number of classes may be used and that the number of classes may differ for different features.

Joint collapse module 310 can include one or more classifiers configured for determining whether a joint is collapsed, such as by determining that a volume of the joint space is insufficient due to not enough pressure provided to the joint space and/or that tissue is blocking the view of the endoscopic imaging device. In one or more examples, the one or more classifiers are configured for determining whether the distal end of the endoscopic imaging device (e.g., distal end 114 of endoscope 103) is outside of a joint. In one or more examples, the one or more classifiers are configured for determining that the joint is clear, which can correspond to the distal end of the endoscopic imaging device being in the joint and there being no tissue blocking the distal end of the endoscopic imaging device and the joint not being collapsed.

The image processing system 300 can transmit the results of the feature determinations performed by the one or more modules to a controller 314 of the surgical pump 312 for the controller 314 to determine if any adjustments to the inflow/outflow or pressure of should be made in light of the characteristics determined at least in part by the one or more classifiers described above with respect to FIG. 3. As described above, adjustments to the surgical pump can include increasing or decreasing the inflow of fluid provided by the surgical pump to a surgical cavity and, in one or more examples, can also include increasing or decreasing the outflow of the surgical pump by for instance increasing or decreasing the rate of suction of the surgical pump. The controller 314 can generate a control command representative of the determined adjustments. In one or more examples, the surgical pump can input the determinations from each of the classifiers and make a determination as to the necessary adjustments to the inflow, outflow, or pressure needed in response to the determined conditions based on the output of each classifier depicted in FIG. 3. In this way, the surgical pump can make decisions as to pressure needs at any given moment during a surgery based on a plurality of conditions that may occur during a surgery as described in further detail below.

In one or more examples, determinations regarding adjustments to settings of the surgical pump are made by the image processing system 300 itself (rather than by the surgical pump) based on the features it determines to be present in the surgical field. The image processing system 300 may then provide instructions to the surgical pump 312 for adjusting its settings. The instructions may include, or be representative of, the control command.

In one or more examples, one or more functions of image processing system 300 described above can be provided by the surgical pump 312 itself. For example, the surgical pump 312 may implement one or more of the image quality module 306, the surgical instrument module 308, and the joint collapse module 310.

In one or more examples, the image processing system 300 or the surgical pump 312 implements a machine learning model controller that is configured to take as input one or more image frames and determine, e.g. a control command representative of, adjustments to surgical pump setting as an output. For example, the image processing system 300 or the surgical pump 312 can implement a deep neural network (DNN) controller generated using model-free reinforcement learning. In one or more examples, the DNN controller can be trained using a trained DNN-based simulator that generates simulated training data sets on which the DNN controller can be trained. The DNN simulator is trained using training data from a physical system, either a physical system operating in real-world scenarios (such as a surgical pump used during a surgical procedure) or a physical system used in a prototyped environment (such as a surgical pump used to control flow through a prototyped surgical cavity). In one or more examples, the machine learning model controller uses inputs other than image frames to determine surgical pump setting adjustments. Examples of inputs that may be used are data from sensors that measure pressure and/or flow rate in the surgical cavity and data from a blood pressure monitor.

The classifiers of the one or more modules of the image processing system 300 can be trained using a supervised training process, an unsupervised training process, a self-supervised training process, a weakly-supervised training process, or a semi-supervised training process. Different classifiers can be trained using a different training process. In a supervised training process, the classifier can be generated by using one or more training images. Each training image can be annotated (i.e., by appending metadata to the image) that identifies one or more characteristics of the image. For instance, using a hip joint classifier configured to identify the presence of a hip joint in an image as an example, the classifier can be generated using a plurality of training images known (a priori) to visualize hip joints.

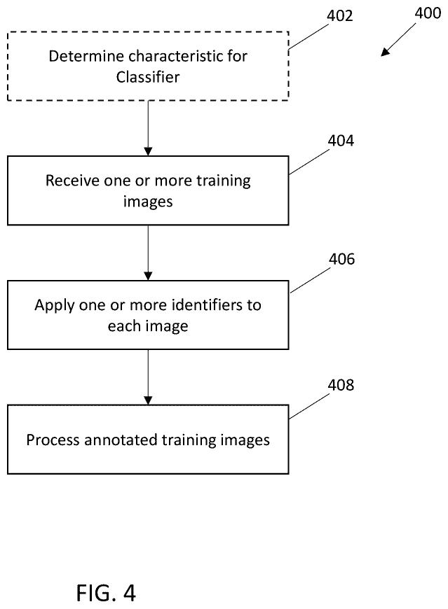

FIG. 4 illustrates an exemplary method for annotating images according to examples of the disclosure. In the example of FIG. 4, the process 400 can begin at step 402 wherein a particular characteristic for a given classifier is selected or determined. In one or more examples, the characteristics can be selected based on the conditions that can influence the inflow, outflow, and/or pressure requirements of a surgical pump during a surgical procedure. Thus, for instance, if a particular medical practice only performs procedures involving hip joints, then the characteristics determined or selected at step 402 will include only characteristics germane to hip surgery contexts. In one or more examples, step 402 can be optional, as the selection of characteristics needed for the classifiers can be selected beforehand in a separate process.

At step 404, one or more training images corresponding to the selected characteristics are received. In one or more examples, each training image can include one or more identifiers that identify the characteristics contained within an image. The identifiers can take the form of annotations that are appended to the metadata of the image, identifying what characteristics are contained within the image. A particular image of the training image set can include multiple identifiers. For instance, a picture of a repaired labrum tear can include a first identifier that indicates the picture contains a hip joint and a separate identifier that indicates the procedure step, which in the example is a repaired labrum. Similarly, a particular training image can include one or more surgical instruments, such as one or more fluid inflow instruments, one or more dedicated suction instruments, and/or one or more types of surgical tools, and the image can be include identifier(s) that indicate the types of surgical instruments in the image.

If the training images received at step 404 do not include identifiers, then the process can move to step 406 wherein one or more identifiers are applied to each image of the one or more training images. In one or more examples, the training images can be annotated with identifiers using a variety of methods. For instance, in one or more examples, the training images can be manually applied by a human or humans who view each training image, determine what characteristics are contained within the image, and then annotate the image with the identifiers pertaining to those characteristics. Alternatively or additionally, the training images can be harvested from images that have been previously classified by a classifier. For instance, and returning to the examples of FIG. 2, once a determination is made as to the features present within an image at step 208, the image can be annotated with the identified feature(s) (i.e., annotated with one or more identifiers) and the image can then be transmitted to and stored in a memory for later use as a training image. In this way, each of the classifiers can be constantly improved with new training data (i.e., by taking information from previously classified images) so as to improve the overall accuracy of the classifier.

In one or more examples, and in the case of segmentation or region based classifiers such as R-CNNS, the training images can be annotated on a pixel-by-pixel or regional basis to identify the specific pixels or regions of an image that contain specific characteristics. For instance in the case of R-CNNs, the annotations can take the form of bounding boxes or segmentations of the training images. Once each training image has one or more identifiers annotated to the image at step 406, the process 400 can move to step 408 wherein the one or more training images are processed by each of the classifiers in order to train the classifier. In one or more examples, and in the case of CNNs, processing the training images can include building the individual layers of the CNN.

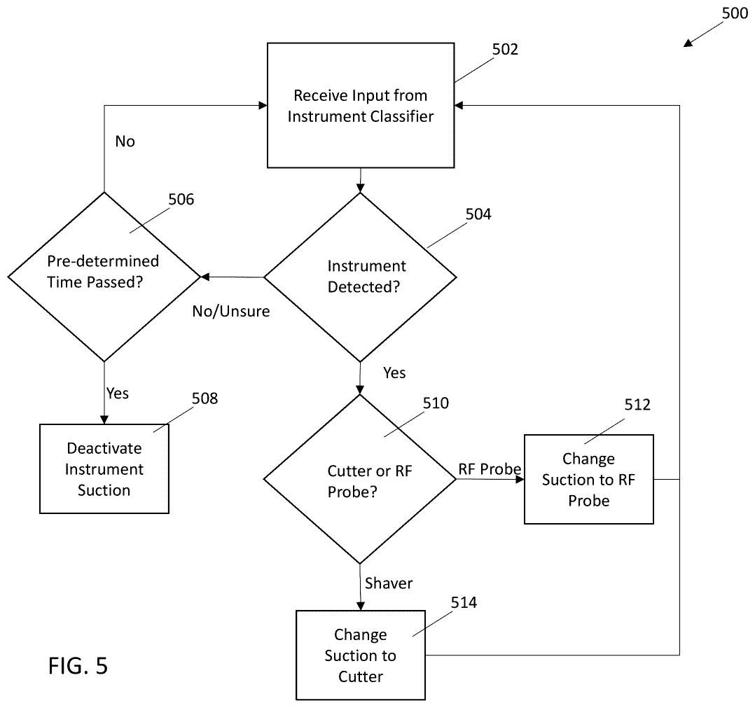

FIG. 5 illustrates an exemplary instrument suction activation process according to examples of the disclosure. The example of FIG. 5 illustrates an exemplary process for adjusting the pressure/flow settings of the surgical pump based on the surgical instruments determined to be present in a given one or more images or video taken from an endoscopic imaging device during a surgical procedure. At step 502, data output by one or more classifiers configured to determine the type of surgical instruments present in a surgical cavity is received by a processor of a surgical pump or a processor that is communicatively coupled to the surgical pump and configured to generate a control command for adjusting, and/or adjust, the flow/pressure settings of the surgical pump. For example, with reference to FIG. 3, surgical pump 312 may receive from the image processing system 300 information regarding the surgical instruments present in one or more image frames as determined by surgical instrument module 308. At step 504, a determination is made as to whether a surgical instrument (associated with the one or more classifiers) is present in the surgical cavity based on the images or video data of the endoscopic imaging device.

In one or more examples, if at step 504 it is determined that a surgical instrument associated with the one or more instrument classifiers was determined to be in the surgical cavity then the process 500 can move to step 510 wherein a determination is made as to which surgical instrument was detected based on the data from the one or more classifiers associated with instrument type. In the example of FIG. 5, the examples of cutters and RF probes are used for illustration. However, the example should not be seen as limiting and can be applied to scenarios in which different and/or additional devices with their own suction are introduced into the surgical cavity. If it is determined at step 510 that an RF probe is present in the surgical cavity (based on the classifier data) then the process 500 can move to step 512 wherein the surgical pump (or a controller communicatively coupled to the surgical pump) can initiate the RF probe's suction, or at least generate a control command thereto. In one or more examples, step 512 can include not only initiating the RF probe's suction but also setting an amount of suction to a level that corresponds to the RF probe (as opposed to a level that corresponds to a different type of surgical instrument). In one or more examples, the initiation of the RF probe's suction can be done prior to the RF probe being activated—i.e., prior to the user activating the RF probe's RF capability. In one or more examples, the RF probe's suction can be ramped up in a controlled fashion such to minimize severe pressure fluctuations in the surgical cavity. In one or more examples, the surgical pump's dedicated suction, such as provided via suction instrument 124 in FIG. 1, can be ramped down in correspondence with ramping up the RF probe's suction such that the pressure and/or flow rate in the surgical cavity is controlled to avoid severe pressure and/or flow rate fluctuations in the surgical cavity. In other words, suction can be handed off from the dedicated suction to the RF probe in a controlled fashion that seeks to reduce severe pressure and/or flow rate fluctuations. In one or more examples, this suction hand-off can be completed prior to activation of the surgical instrument so that when the user activates the surgical instrument, some or all of the suction is already being provided by the surgical instrument, which avoids an abrupt change in pressure that would occur if, for example, suction were switched upon instrument activation.

In one or more examples, the ramping up of suction of the RF probe and ramping down of the suction of the dedicated suction continues until a desired balance between suction provided by the various suction sources is achieved. For example, a balance can be 50% of the suction being provided by the dedicated suction and 50% being provided by the RF probe, or a balance can be all suction being provided by the RF probe and none by the dedicated suction. These balances are merely exemplary and it should be understood that any balance of suction between the RF probe, dedicated suction, and/or other suction sources present in the surgical cavity can be used.

The balance between suction provided by the RF probe (or other surgical instrument) and suction provided by a dedicated suction can be a dynamic balance that may be adjusted based on conditions of the fluid flow in the surgical cavity and/or the surgical instrument/dedicated suction at a given time. For example, a suction provided by the RF probe may be increased to a desired level (e.g., a maximum level achievable by the RF probe) and a suction provided by the dedicated suction may be decreased to level needed to achieve a total outflow for a desired flow rate/and or pressure in the surgical cavity, which could be different for different anatomy, different stages of surgery, etc. This could be useful, for example, in situations where the suction provided by the RF probe alone is insufficient to achieve the desired outflow, such as due to the RF probe having a smaller suction conduit than the dedicated suction, a manual valve on the RF probe not being open (or not being fully open), and/or clogging of the RF probe suction pathway. In one or more examples, the balance can be based on one or more additional characteristics associated with the procedure that are determined from the images or video data captured by the endoscopic imaging device, such as the joint type, joint condition (e.g., collapsed or partially collapsed state), and/or clarity. For example, where a high flow rate is desired for improving clarity, a combination of suction from the RF probe and dedicated suction may be used, whereas where a lower flow rate is desired, suction provided only by the RF probe may be sufficient.

In one or more examples, the amount of suction provided by one or more suction sources can be adjusted as conditions change. For example, in response to detecting one or more features in one or more image frames indicating conditions that affect clarity, one or more aspects of the fluid inflow to the surgical cavity may be adjusted, or a control command may be generated, to increase fluid flow and/or pressure and, correspondingly, a suction provided by the surgical instrument and/or the dedicated suction may be increased. Similarly, in one or more examples, in response to detecting the collapse of the surgical cavity (or partial collapse of the cavity), the amount of suction provided by the surgical instrument and/or the dedicated suction may be decreased to increase the pressure in the surgical cavity. In one or more examples, suction provided by the surgical instrument may be decreased in the event of joint collapse only if a determination is made that the surgical instrument is active. In addition to decreasing suction or alternatively to decreasing suction, fluid inflow pressure and/or fluid inflow flow rate may be increased to prevent collapse of the joint and/or re-expand the joint. The surgical pump (or a controller communicatively coupled to the surgical pump) may be configured to determine that a surgical instrument is active by receiving a communication from the surgical instrument or the system comprising the surgical instrument indicating that the surgical instrument is active. For example, the RF driver for the RF probe may output a signal to the surgical pump or a system component communicatively coupled to the surgical pump indicating that the RF probe is active. Alternatively, one or more classifiers may be configured to detect that an instrument is active, such as by detecting rotation of a cutting tool from the images and/or video data captured by the endoscopic imaging device.

Conditions that trigger, or are otherwise associated with, an adjustment in or amount of suction provided by the suction source(s) need not be limited to conditions associated with features detected in one or more image frames. In one or more examples, the surgical pump may be capable of detecting clogging of a suction source in the surgical cavity and, in response, adjust the amount of suction provided by another suction source in the surgical cavity. For example, the surgical pump may detect that the RF probe suction pathway has become clogged (e.g. by detecting a pressure drop in the fluid pathway associated with the RF probe suction) and may respond by ramping up suction provided by the dedicated suction instrument.

In one or more examples, a condition that may trigger an adjustment in (or otherwise be associated with an amount of) suction provided by one or more suction sources in the surgical cavity may be a deactivation of a surgical instrument supplying suction in the surgical cavity. The surgical pump may be configured to determine that a surgical instrument has been deactivated by receiving a communication from the surgical instrument or the system comprising the surgical instrument. For example, the RF driver for the RF probe may output a signal to the surgical pump or a system component communicatively coupled to the surgical pump indicating that the RF probe has been deactivated and, in response, the surgical pump may reduce a suction supply to the RF probe. Alternatively, one or more classifiers may be configured to detect that an instrument is no longer active, such as by detecting from the images that rotation of a cutting tool has ceased. Optionally, after determining that a surgical instrument has been deactivated, a determination may be made whether the surgical instrument is present in the surgical cavity. In response to a determination that the surgical tool is present, the suction may be reduced but not deactivated. In some examples, the suction may be maintained at the same level. If the surgical instrument is determined to not be present (according to the steps described below), the suction to the surgical instrument may be deactivated altogether.

In one or more examples, a condition that may trigger an adjustment in (or otherwise be associated with an amount of) suction provided by one or more suction sources in the surgical cavity and/or an adjustment to one or more characteristics of fluid inflow to the surgical cavity is a change in blood pressure. For example, if blood pressure (e.g., mean arterial pressure) is determined to be higher than a pressure in the surgical cavity, suction through one or more instruments (e.g., an RF probe and/or a dedicated suction instrument) could be decreased to increase the pressure in the cavity in order to prevent or reduce bleeding into the surgical cavity. In one or more examples, one or more characteristics of fluid inflow to the surgical cavity can be adjusted without adjusting suction or in combination with adjusting suction to raise the cavity pressure in the event the cavity pressure is lower than the blood pressure. In one or more examples, the detection of one or more characteristics in the imaging may trigger adjustments to the inflow flow characteristics and/or the suction that are based on the blood pressure. For example, the surgical pump may receive an indication from an image processing system that blood has been detected in the imaging, such as via one or more steps of process 700 or process 800 described below, the surgical pump may respond by comparing the cavity pressure to the blood pressure (e.g., as received by the surgical pump from a blood pressure monitoring system) and may (generate a control command to) automatically increase the cavity pressure to a level that is above the blood pressure if the determination is made by the surgical pump that the blood pressure is higher than the cavity pressure. Blood pressure information can be provided to the surgical pump by a blood pressure monitoring system that is communicatively coupled to the surgical pump. In some examples, the system can decrease the inflow parameters (e.g., pressure, inflow rate) if the joint pressure is higher than the blood pressure measured by the blood pressure monitor and/or if the difference between the joint pressure and the blood pressure exceeds a predefined threshold.

Similar to the process for the RF probe described above, if it is determined at step 510 that a cutter is present in the surgical cavity then the process 500 can move to step 514 wherein the surgical pump/controller can (generate a control command to) initiate the cutter's suction prior to activation of the cutter, and/or deactivate or ramp down the surgical pump's dedicated suction. In one or more examples, step 514 includes setting an amount of suction based on the cutter being detected. In one or more examples, after both steps 512 and 514, the process 500 can revert back to step 502 so that the system can detect when the surgical instrument has been removed (as to be further described below). Although the steps above are described with respect to a cutter and an RF probe, it should be understood that these same steps can be used for any other instruments that have suction capability and that can be determined to be present in one or more image frames by the one or more classifiers.

In one or more examples, if at step 504 it is determined that no instrument is in the surgical cavity or if the classifier is unsure that an instrument is in the surgical cavity (for instance if the prediction is halfway between 0 and 1) the process 500 can move to step 506 wherein a determination is made as to whether a pre-determined time has passed since the classifiers began to not detect an instrument or were unsure that an instrument was present. In one or more examples, when an instrument is present in the surgical cavity but then “disappears” from the classifiers (i.e., the classifiers no longer see the surgical instrument in the images), the disappearance may be caused by a momentary error in the classifier or because the surgical instrument has been removed by the surgeon from the surgical cavity. If the disappearance is caused by a momentary error, reacting to that error by adjusting the surgical pump could propagate the error and cause an improper amount of pressure to be delivered via the surgical pump to the surgical cavity. Thus, in one or more examples, the process 500 can wait a pre-determined amount of time after an instrument disappears from the classifiers before adjusting the pressure or pressure setting to account for the removal of the surgical instrument. At step 506, in one or more examples, the first time an instrument disappears from the classifiers, a timer can be started and the process can revert back to step 502 to receive additional data from the one or more instrument classifiers. Each time no instrument is determined to be present in an image frame at step 504, the process can go to step 506 to check if the pre-determined time has passed. If not, then the process again reverts back to step 502, thus creating a loop that is broken when an instrument is determined to be in the surgical cavity, or when the pre-determined time has passed since the disappearance of the surgical instrument from the classifiers.

In one or more examples, once the pre-determined time has passed at step 506 the process 500 can move to step 508, wherein suction from the surgical instrument that was removed is deactivated. In one or more examples, suction from a dedicated suction of the surgical pump (i.e., suction instrument 124 of surgical pump 120 shown in FIG. 1) is increased (increased from a lower level to a higher level or increased from zero) such that suction is maintained in the surgical cavity, or at least a control command the generated thereto.

In one or more examples, one or more characteristics of fluid inflow to the surgical cavity can be adjusted by the surgical pump in accordance with adjustments made to the suction in the surgical cavity. For example, enabling and/or disabling of suction by a surgical instrument may cause fluctuations in fluid pressure in the surgical cavity that may be corrected for by adjusting one or more characteristics of fluid inflow provided by the surgical pump, such as by adjusting the surgical pump flow rate and/or the surgical pump head pressure.

The determination that one or more surgical instruments is present in one or more image frames can additionally or alternatively include determining the presence of one or more fluid inflow instruments (e.g., inflow instrument 122 of FIG. 1) in the surgical cavity, which can be used to start, stop, and/or adjust fluid flow provided by the inflow instrument. In one or more examples, a fluid inflow instrument is visible in one or more image frames and one or more machine learning models, such as one or more machine learning models of surgical instrument module 308 of FIG. 3, can be configured to identify the fluid inflow instrument in the one or more image frames. In one or more examples, a fluid inflow instrument is attached to or otherwise integrated with the endoscope (e.g., endoscope 103 of FIG. 1) such that it is not visible in image frames, and a determination that the endoscope is in the surgical cavity is used as a proxy for the fluid inflow instrument being in the surgical cavity. For example, with reference to FIG. 3, joint collapse module 310 can be configured to determine whether the endoscope is inside or outside of the surgical cavity and this determination can be used by the surgical pump to adjust fluid inflow.

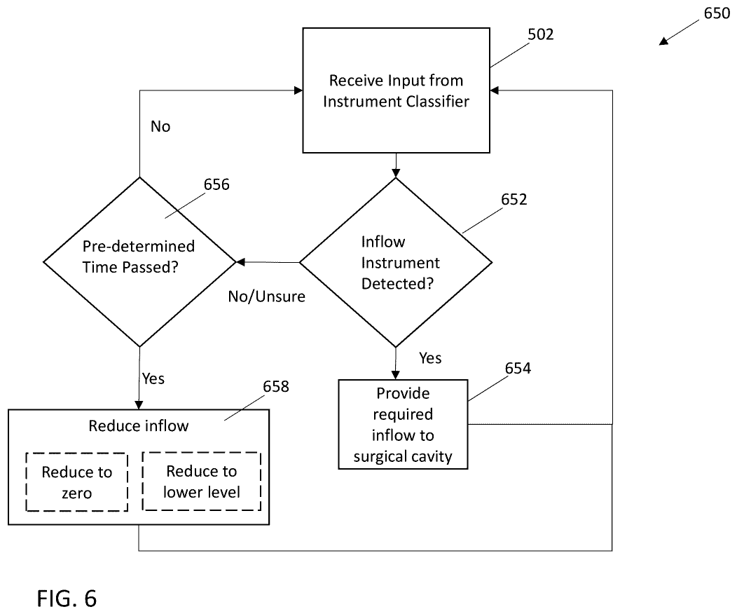

The process for adjusting fluid inflow based on the detection of the fluid inflow instrument in the surgical cavity can be similar to process 500. A flow diagram of an example of such a process is shown in FIG. 6. Process 650 can be performed in combination with process 500. For example, one or more steps of process 650 can be combined with one or more steps of process 500. In one or more examples, process 650 is performed but not process 500. Process 650 can be performed by one or more processors of the surgical pump or by one or more processors communicatively connected to the surgical pump.

Process 650 can begin with the same step 502 of process 600—data output by one or more classifiers configured to determine the type of surgical instruments present in a surgical cavity is received by a processor of a surgical pump or a processor that is communicatively coupled to the surgical pump and configured to adjust the flow/pressure settings of the surgical pump. At step 652, a determination is made whether a fluid inflow instrument is present in the surgical cavity based on the image frames or video data from the endoscopic imaging device (e.g., by identifying the fluid inflow instrument in the image frames or video data or by determining that the endoscope is inside of the surgical cavity). If the determination is made that the fluid inflow instrument is present in the surgical cavity, then at step 654, (a control command can be generated such that) the inflow required for the surgical cavity can be provided by the fluid inflow instrument, such as per process 200. Optionally, step 654 can include automatically initiating fluid inflow when a determination is made that the inflow instrument has just appeared in the surgical cavity. If the determination is made in step 652 that the inflow instrument is not present in the images, then at step 656, a determination is made as to whether a pre-determined time has passed since the classifiers began to not detect the inflow instrument or were unsure that the inflow instrument was present, in similar fashion to step 506 of process 600. At step 658, once a predetermined time has passed since the fluid inflow device was last detected, the surgical pump can reduce the fluid inflow provided via the inflow instrument. In one or more examples, the inflow flow rate can be reduced to zero (deactivated altogether) such that there is no flow provided by the inflow instrument. In one or more examples, the inflow flow rate can be reduced to a lower level. This may be desirable, for example, to enable the surgeon to use the reduced flow from the inflow device outside of the surgical cavity, such as to rinse and area of tissue or one or more other instruments. The steps of process 650 can be repeated such that, for example, flow provided by the inflow instrument is increased upon reentry of the inflow instrument to the surgical cavity (as determined from the images or video data).

As described above with respect to FIG. 3, the system can include one or more image clarity classifiers. As described above, various conditions that can inhibit the clarity of a video such as blood, bubbles, debris, snow globe conditions, and turbidity, if detected, can require a change to the pressure and/or flow settings of the surgical pump. Thus, in one or more examples, the one or more classifiers can be configured to determine these conditions. In one or more examples, each clarity condition (i.e., blood, bubbles, turbidity, snow globe, debris) can be detected by its own classifier. In one or more examples, multiple clarity conditions can be detected by a single classifier.

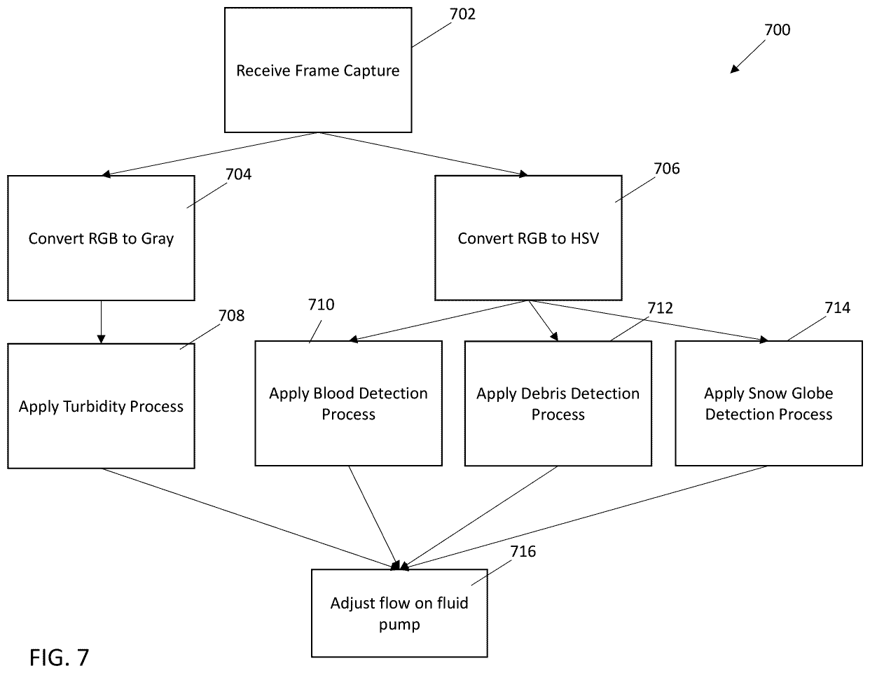

FIG. 7 illustrates an exemplary image clarity based process for controlling a surgical pump according to examples of the disclosure. The example of FIG. 7 illustrates a process 700 that takes as its input one or more images captured from an endoscopic imaging device video feed, and processes them to identify one or more types of visual disturbances present in the images, and uses the information to adjust the inflow/outflow or pressure settings of the surgical pump. In one or more examples, the process 700 can begin at step 702 wherein one or more captured image frames from an endoscopic imaging device video feed are received. Once the captured frames are received at step 702, each frame can be converted from a conventional red, green, blue (RGB) color space to one or more alternative color spaces that are configured to accentuate various visual phenomenon that can affect the clarity of a given image. Thus, in one or more examples, after receiving the captured image frames at step 702, the process 700 can simultaneously and in parallel convert a single image into two separate images with a modified color space as depicted at steps 704 and 706.

In one or more examples, at step 704, the one or more images received at step 702 can be converted from the RGB color space to the Grayscale color space. In the grayscale color space, each pixel rather than representing a particular color can instead represent an amount of light (i.e., an intensity). Converting an image from RGB to grayscale as described in further detail below can accentuate various features of the image that make it easier to identify certain visual phenomenon such as turbidity.

In one or more examples, at step 706, the one or more images received at step 702 can be converted from the RGB color space to the hue, saturation, value (HSV) color space. The HSV color space can describe colors in terms of their shade (i.e., amount of gray) and their brightness value). Converting an image from the RGB color space to the HSV color space can also be used to accentuate various features of the image that make it easier to identify certain visual phenomenon such as blood, debris, and a snow globe effect (described in further detail below). In one or more examples, after converting the one or more image from RGB to HSV at step 706, the process 700 can apply one or more image processing algorithms to the converted images to identify specific visual phenomenon (described in further detail below) as depicted in steps 710, 712, and 714.

In one or more examples, at step 710, the process 700 can apply a blood detection process to the converted image to detect the presence of blood in a given image. As described in further detail below, while some blood is to be expected during a surgical procedure, an excess amount of blood can create a visual impairment for the surgeon during a surgery and thus the surgical pump may need to be adjusted to apply more pressure in the surgical cavity so as to arrest or minimize the amount of blood present in the surgical cavity. In one or more examples, at step 712, the process 700 can apply a debris detection process to the converted image to detect the presence of debris in a given image. Debris can refer to particles in the surgical cavity that are unnecessary and can be caused by loose fibrous tissue or resected tissue/bone floating in the joint space fluid. In one or more examples, at step 714, the process 700 can apply a snow globe detection process to the converted image. In one or more examples, a “snow globe” effect can refer to debris generated by resecting bone that causes poor visibility in the joint space. Thus, at step 714, the snow globe detection process using the HSV color space image can perform an algorithm (described in further detail below) that can be used to identify a snow globe effect.

Referring back to step 704, the grayscale image can also be used to identify one or more visual phenomenon. For instance, in one or more examples of the disclosure, once an image has been converted from RGB to grayscale at step 704, the process 700 can move to step 708 wherein the grayscale image is used to determine the turbidity present in the image. In one or more examples, turbidity can refer to the cloudiness or haziness of a fluid caused by particles floating in a liquid medium. Thus, at step 708, an algorithm (described in detail below) can be applied to a grayscale image to determine turbidity levels in the image. Once each of the processes depicted at steps 708, 710, 712, and 714 have been performed, the process 700 can move to step 716 wherein the inflow, outflow, and/or pressure settings of the surgical pump can be adjusted based on the outcomes of the processes.

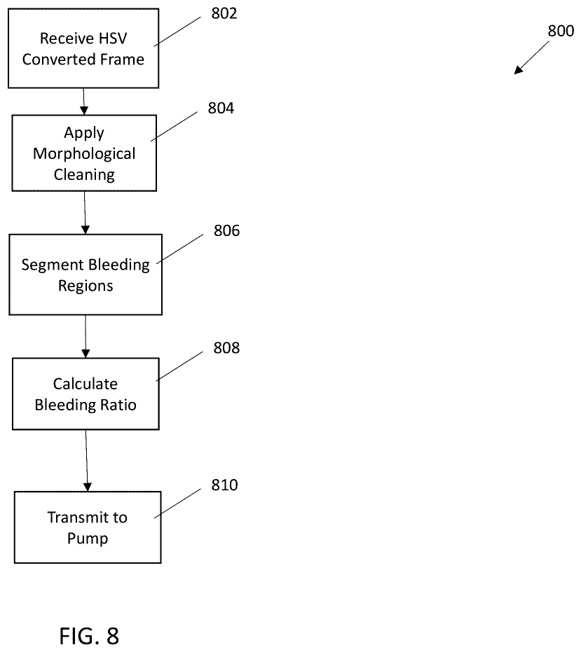

FIG. 8 illustrates an exemplary process for detecting blood in an image according to examples of the disclosure. In one or more examples, the process 800 can begin at step 802 wherein an HSV converted image frame (described above with respect to step 706 of FIG. 7) is received. In one or more examples, after the HSV converted image frame is received at step 802, the process 800 can move to step 804 wherein a morphological cleaning process is applied to the image. In one or more examples, a morphological cleaning process can refer to an image processing algorithm that can be applied to an image to grow or shrink image regions as well as remove or fill-in image region boundary pixels. The morphological cleaning process can be configured to enhance image regions (such as regions in which bleeding is present) so that they can be more easily identified.

After morphological cleaning is applied to the image at step 804, the process 800 can move to step 806 wherein one or more bleeding regions are segmented within the image. A “bleeding region” can refer to a region in the image in which blood is present. In one or more examples, a bleeding region can be identified based on the HSV characteristics of the pixels (i.e., pixels that contain HSV values that are indicative of blood). For instance, a bleeding or bleed region can be identified based on pixels that are within a certain range of HSV values. In one or more examples, segmenting the image can refer to identifying regions or segments in the image in which, based on the HSV values, blood is likely present. Once the bleeding regions have been segmented at step 806, the process 800 can move to step 808 wherein a ratio of the area covered by bleeding regions over the total area shown in the image is calculated. This ratio can represent how much blood is contained in a given image as a function of the percentage of space of the total image area occupied by bleeding regions. Thus, as an example, if a total image area is 100 pixels and the sum of all the bleeding regions occupies only 3 pixels then the ratio can be determined to be 3%, meaning that the bleeding regions occupy 3% of the total image area.

Once the ratio has been calculated at step 808, the process 800 can move to step 810 wherein the calculated ratio is transmitted to the surgical pump or a controller communicatively coupled to the surgical pump that can generate a control command for adjusting, and/or adjust, the flow settings of the surgical pump based on the determined ratio. The pre-determined threshold, in one or more examples, can be empirically determined. Additionally or alternatively, the pre-determined threshold can be set based on the surgeon's preferences. In one or more examples, the surgical pump can increase the pressure settings if the calculated ratio is greater than a pre-determined threshold. For instance, if the ratio is found to be 30% while the pre-determined threshold is 50% then the surgical pump may take no action and leave the pressure settings of the surgical pump as is. However, if during the surgery the ratio increases to 60%, then the surgical pump may increase the pressure in an attempt to minimize or stop the bleeding in the surgical cavity. In one or more examples, the surgical pump or a controller communicatively coupled to the surgical pump can generate the control command to increase, and/or increase, the pressure in a time-based manner. For example, if the determined ratio meets or exceeds the pre-determined threshold, a timer can be initiated to control the rate of increasing the pressure in the joint. In one or more examples, the rate of increase can be based on the period of time that a visual disturbance is detected. For instance, the longer blood is detected in the joint, the faster the pressure increases (i.e., the rate increases). In one or more examples, the rate of increase can reset to zero when it is determined that there is no longer a visual disturbance, or only a minimal amount of visual disturbance.

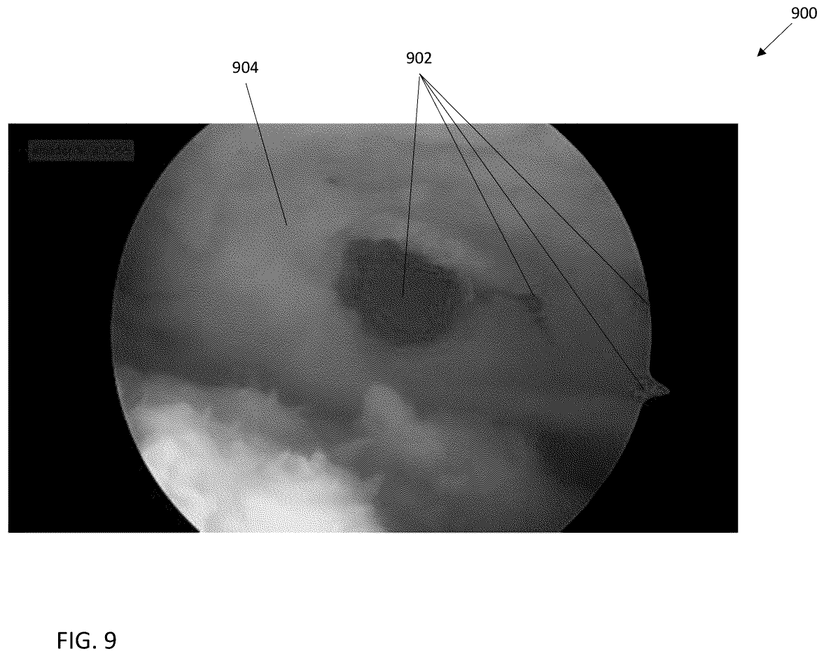

FIG. 9 illustrates an exemplary endoscopic image with segmented bleed regions according to examples of the disclosure. In the example of FIG. 9, the image 900 can include one or more bleed regions 902 as identified at step 806 in the example of FIG. 8. The example of FIG. 9 shows an image that contains a 3% bleed ratio, meaning that the identified bleed regions occupy about 3% of the total scope area.

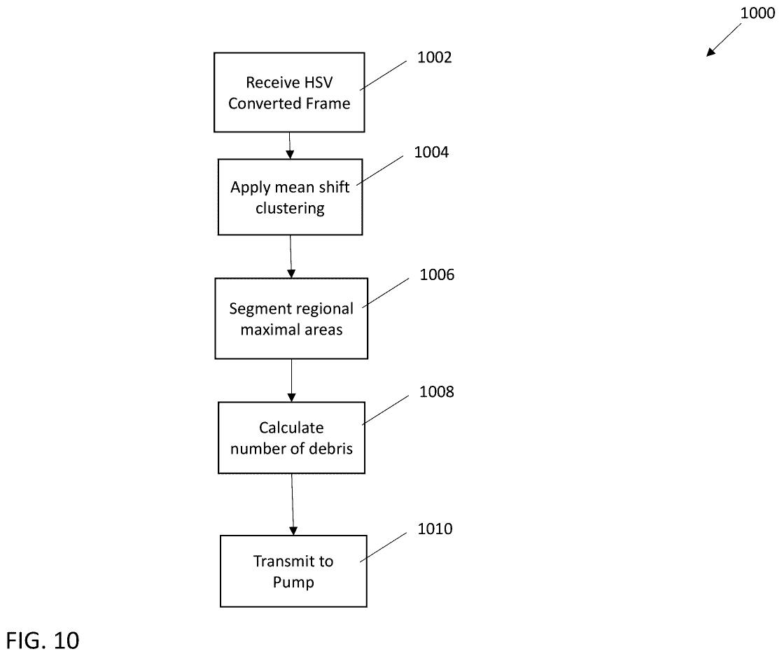

FIG. 10 illustrates an exemplary process for detecting debris in an image according to examples of the disclosure. In one or more examples, the process 1000 can begin at step 1002 wherein an HSV converted image frame (described above with respect to step 706 of FIG. 7) is received. With respect to debris, the HSV color space can make it easier to distinguish debris (i.e., loose fibrous tissue floating in the internal area) from other tissue and objects that are imaged in a surgical cavity. As described above, this debris can represent visual impairments to a surgeon when performing a surgical procedure, and thus in order to automate the process of adjusting the pressure and/or outflow to remove or minimize debris the process should be able to automatically distinguish debris from other matter in the surgical cavity.

In one or more examples, after the HSV converted image frame is received at step 1002, the process 1000 can move to step 1004 wherein a mean shift clustering algorithm is applied to the received image frame. In one or more examples, the mean shift clustering algorithm can be configured to locate the local maxima of an image given data sampled from the image (i.e., the pixel values). In one or more examples, the debris in an image will appear as small areas in an image where the pixel values suddenly shift. The mean shift clustering algorithm can identify the areas in an image where the mean pixel values suddenly shift (i.e., local maxima) thus identifying individual pieces of debris in a given image.

Once the mean shift clustering algorithm is applied at step 1004, the process 1000 can move to step 1006 wherein the regional maximal areas/regions are segmented from the image. In one or more examples, each regional maximal area can represent a piece of debris in the image. Thus by identifying these regions, and as described below, the process 1000 can calculate the specific number of debris pieces that are found within a given image. Once the regions have been segmented at step 1006, the process 1000 can move to step 1008 wherein the number of pieces of debris in a given image are counted. In one or more examples, counting pieces of debris can include simply counting the number of regional maximal areas identified in step 1006. Finally, at step 1010, the number of debris can be transmitted to the surgical pump or a controller communicatively coupled to the surgical pump so as to generate a control command for adjusting, and/or adjust, the pressure settings of the surgical pump based on the number of pieces of debris found in an image.

In one or more examples, the surgical pump can be adjusted by increasing an amount of suction (i.e., outflow) that the surgical pump is generating. By increasing the suction, the debris in a surgical cavity can be removed at a quicker rate to thereby remove the overall amount of debris in a surgical cavity and thereby removing or minimizing the visual impairments to the surgeon. In one or more examples, the amount of suction can be based on the number of pieces of debris found in the surgical cavity based on the images and/or video data captured from the endoscopic imaging device. In one or more examples, the surgical pump can also adjust the inflow of the fluid to sweep the debris out of the visualized area.

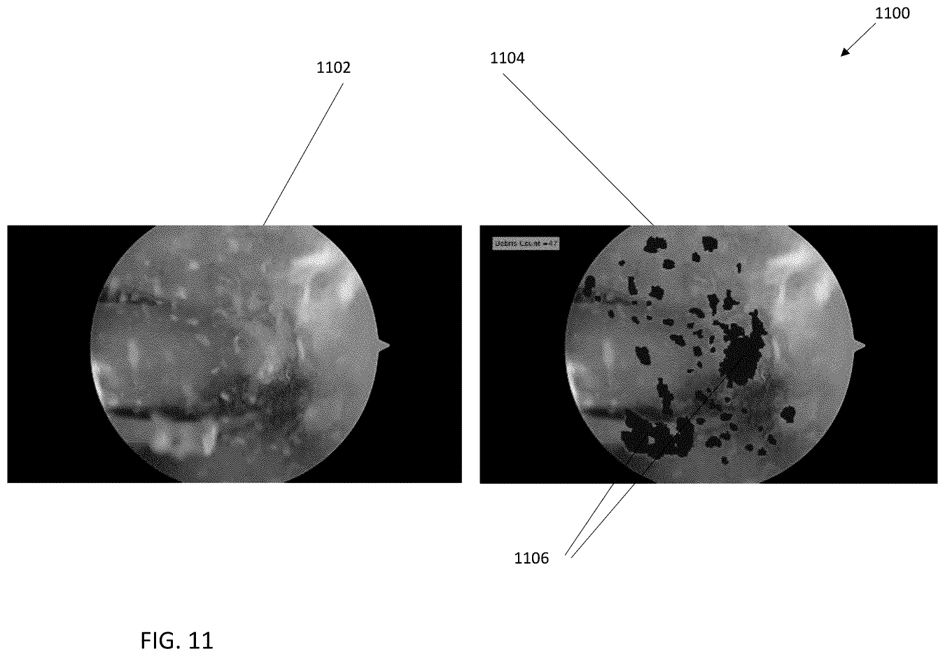

FIG. 11 illustrates an exemplary endoscopic image with identified debris clusters according to examples of the disclosure. The images 1100 of FIG. 11 can include a first image 1102 that illustrates an image with debris that has not been processed to identify the individual pieces of debris. Thus, the image 1102 illustrates an image with debris before the process described above with respect to FIG. 10 is applied to the image. The images 1100 include a second image 1104 that shows the identified debris pieces 1106 once the process described above with respect to FIG. 10 is applied to the image.

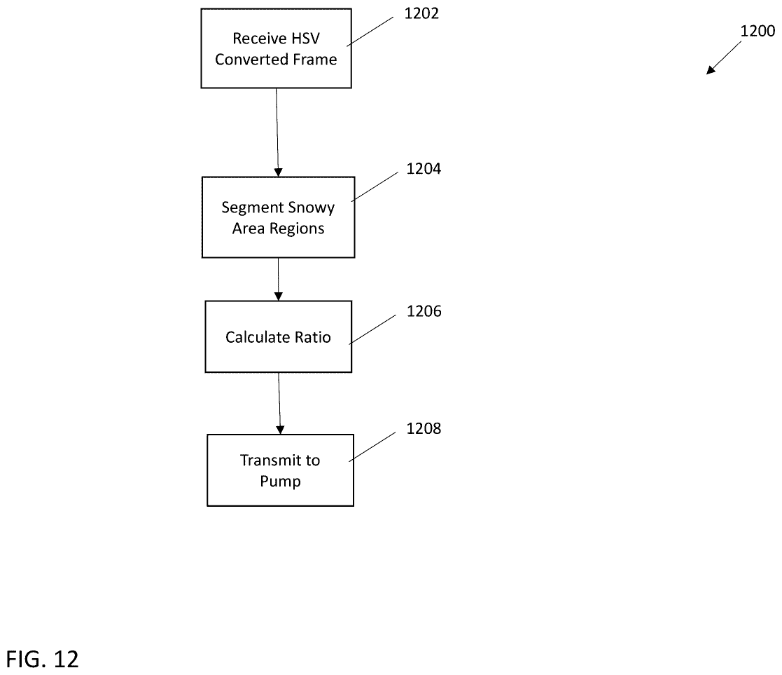

FIG. 12 illustrates an exemplary process for detecting a snow globe effect in an image according to examples of the disclosure. In one or more examples, the process 1200 can begin at step 1202 wherein an HSV converted image frame (described above with respect to step 706 of FIG. 7) is received. In one or more examples, after the HSV converted image frame is received at step 1202, the process 1200 can move to step 1204 wherein one or more snowy area regions are segmented within the image. A “snowy area region” can refer to a region in the image in which the snow globe effect (i.e., debris from resected bone) is present. In one or more examples, a snowy area region can be identified based on the HSV characteristics of the pixels (i.e., pixels that contain HSV values that are indicative of a snow globe effect). For instance, a snow globe region can be identified based on pixels that are within a certain range of HSV values. In one or more examples, segmenting the image can refer to identifying regions or segments in the image in which, based on the HSV values, the snow globe effect is likely present. Once the snowy area regions have been segmented at step 1204, the process 1200 can move to step 1206 wherein a ratio of the area covered by snowy area regions over the total area shown in the image is calculated. This ratio can represent how prevalent the snow globe effect is in a given image as a function of the percentage of space of the total image area occupied by snowy area regions. Thus, as an example, if a total image area is 100 pixels and the sum of all the snowy area regions occupies only 3 pixels then the ratio can be determined to be 3%, meaning that the snowy area regions occupy 3% of the total image area.

Once the ratio has been calculated at step 1206, the process 1200 can move to step 1208 wherein the calculated ratio is transmitted to the surgical pump or a controller communicatively coupled to the surgical pump that can determine an adjusted the flow settings of the surgical pump based on the determined ratio. The surgical pump or the controller communicatively coupled to the surgical pump can generate a control command representative of the adjusted the flow settings. In one or more examples, the surgical pump can increase the pressure settings if the calculated ratio is greater than a pre-determined threshold. For instance, if the ratio is found to be 30% while the pre-determined threshold is 50% then the surgical pump may take no action and leave the pressure settings of the surgical pump as is. However, if during the surgery the ratio increases to 60%, then the surgical pump may increase the pressure in an attempt to minimize or remove the debris from resected bone in the surgical cavity. The pre-determined threshold, in one or more examples, can be empirically determined. Additionally or alternatively, the pre-determined threshold can be set based on the surgeon's preferences. In one or more examples, rather than increasing the pressure, the surgical pump can be adjusted to increase the suction so as to remove the resected bone that is causing the snow globe effect.

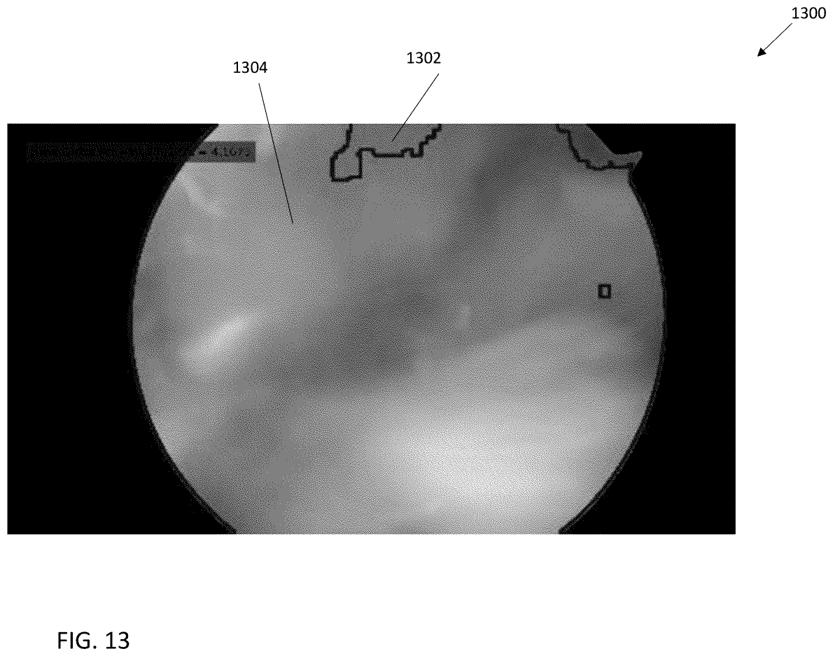

FIG. 13 illustrates an exemplary endoscopic image with segmented snowy area regions according to examples of the disclosure. In the example of FIG. 13, the image 1300 can include one or more snowy area regions 1304 as identified at step 1204 in the example of FIG. 12. In one or more examples, the snowy area regions can be distinguished from other regions 1302 where the snow globe effect is not present.

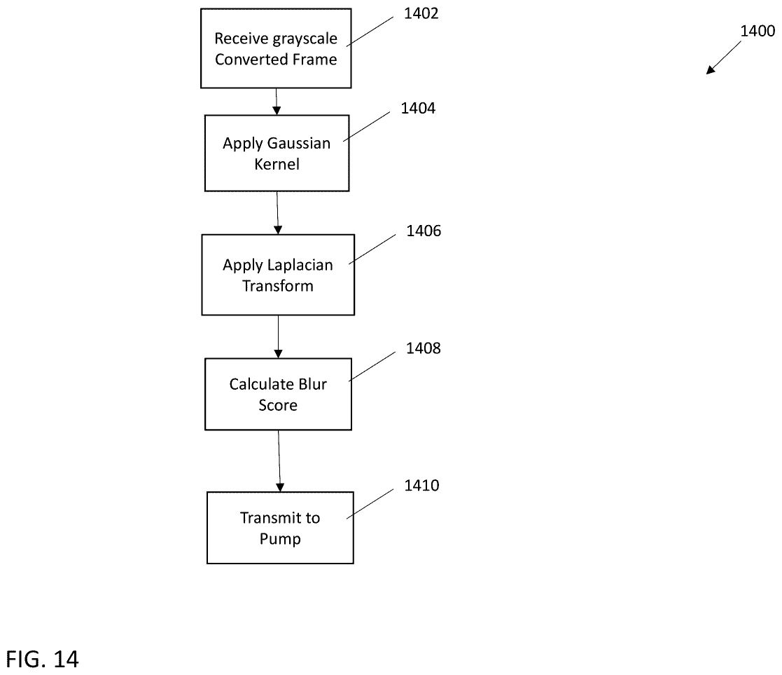

FIG. 14 illustrates an exemplary process for detecting turbidity in an image according to examples of the disclosure. In one or more examples, the process 1400 of FIG. 14 can begin at step 1402 wherein a grayscale converted image is received as described above with respect to step 714 of FIG. 7. Once the grayscale image is received at step 1402, the process 1400 can move to step 1404 wherein the image is convolved with a Gaussian kernel. Convolving the image with a Gaussian kernel at step 1404 can suppress the noise in the image, to allow for further image processing. Once the Gaussian kernel is applied at step 1404, the process 1400 can move to step 1406 wherein a Laplacian transform is applied to the image. The Laplacian transform can be used to find areas of rapid change (edges) in the image.

Once the Laplacian transform is applied at step 1406, the process 1400 can move to step 1408 wherein a blur score is calculated from the result of step 1406. In one or more examples, the blur score can represent the degree of blur in the image. A high blur score can indicate that the image is blurry and can therefor indicate the presence of turbidity in the image. A low blur score can indicate the absence of turbidity. Once the blur score has been calculated at step 1408, the process 1400 can move to step 1410 wherein the blur score is transmitted to the surgical pump or a controller communicatively coupled to the surgical pump.

The pressure or inflow/outflow settings of the surgical pump can be adjusted based on the calculated blur score. In one or more examples, the blur score calculated at step 1408 can be compared against a pre-determined threshold to determine if the surgical pump needs to be adjusted based on the blur score. In one or more examples, if the blur score is higher than the pre-determined threshold then the surgical pump can generate a control command to take action, and/or take action, to increase the pressure (described in further detail below). The pre-determined threshold, in one or more examples, can be empirically determined. Additionally or alternatively, the pre-determined threshold can be set based on the surgeon's preferences. In one or more examples, the inflow of the surgical pump can be pulsed to keep stagnant fluid away from the scope.

As described above, each of the individual clarity classifiers described above with respect to FIGS. 7-14 can individually cause the surgical pump to increase or decrease the pressure settings by increasing or decreasing the inflow/outflow or by increasing and decreasing the suction of the surgical pump. In one or more examples, the clarity classifiers can also collectively cause an adjustment to the surgical pump pressure settings.

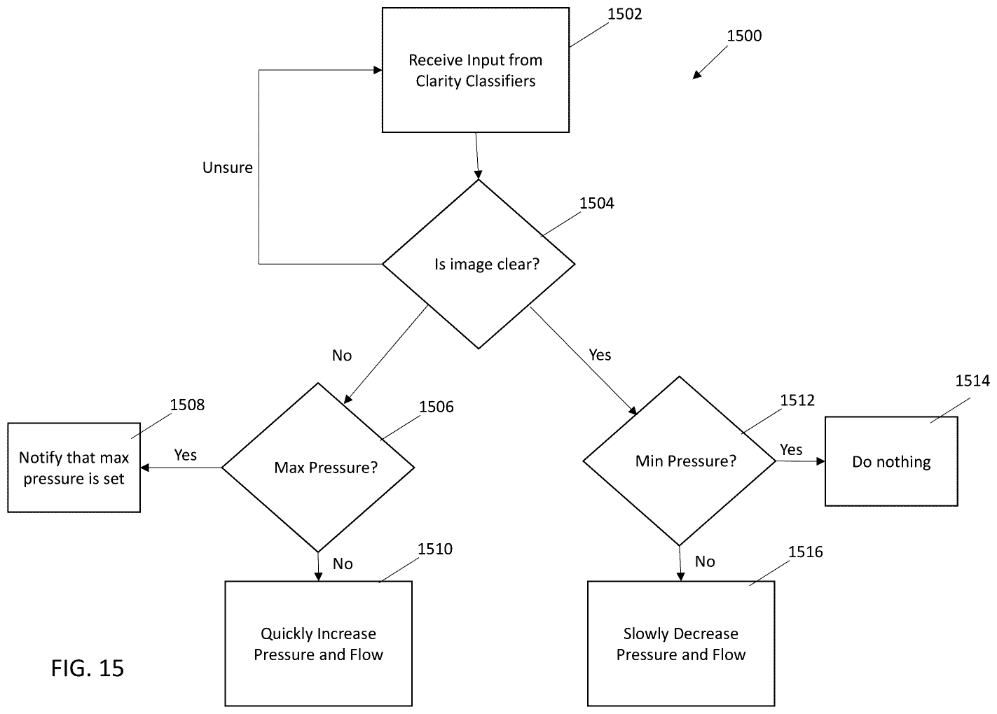

FIG. 15 illustrates an exemplary process for adjusting the settings of a surgical pump based on the image clarity according to examples of the disclosure. In one or more examples, the process 1500 of FIG. 15 can begin at step 1502 wherein the data from each clarity based classifier is received. The data can represent the output values of each classifier that is transmitted to the surgical pump or a controller communicatively coupled to the surgical pump as described above. Once the inputs are received at step 1502, the process 1500 can move to step 1504 wherein a determination is made as to whether the image is clear. As described above, the determination can be based on whether the outputs of the classifiers are greater than or less than a pre-determined threshold. In one or more examples, if one of the outputs of the classifiers is greater than its corresponding pre-determined threshold, then it can be determined that the image is not clear. In one or more examples, if a certain number of classifier outputs are higher than their corresponding pre-determined thresholds, then the process 1500 at step 1504 can determine that the image is not clear. In one or more examples, if a plurality of outputs are greater than their corresponding pre-determined thresholds, and a plurality of outputs are less than their corresponding pre-determined thresholds, then the process 1500 at step 1504 can determine that it is unsure about the clarity of the image.

In one or more examples, if the process 1500 at step 1504 determines that it is unsure about the image, then the process 1500 can do nothing with respect to the pressure settings of the surgical pump and revert back to step 1502 of process 1500 to receive further data from the one or more clarity based classifiers. A determination of unsure can mean that it is not apparent that there is a visual disturbance, and so, rather than change the settings of the surgical pump, the process can instead do nothing and wait for more data.

In one or more examples, if the process 1500 at step 1504 determines that the image is not clear, then the process 1500 can move to step 1506 wherein the process 1500 can determine if the surgical pump is at a maximum allowable pressure. As described above, if an image is not clear, then the surgical pump may need to take one or more actions to increase the pressure in the surgical cavity to remove or minimize one or more visual disturbances that are causing the image to not be clear. For example, the surgical pump may generate a control command to decrease, and/or decrease, the suction provided by one or more surgical instruments in the surgical cavity, which may increase pressure in the surgical cavity. However, as also described above, there exists a maximum pressure setting for the surgical pump that if exceeded could cause injury or damage to the patient. This pressure level can be context dependent. For instance, the maximum allowable pressure for a knee surgery may be different than the maximum allowable pressure for a shoulder surgery. Thus, while a determination that the image is not clear may require the pressure exerted by the surgical pump to increase, a check is first done at step 1506 to make sure that the surgical pump is not already at its maximum allowable pressure settings for the area in which the surgery is occurring (or other factors that can influence the maximum allowable pressure).

In one or more examples, a determination that a maximum allowable pressure has been reached may be made based on analysis (e.g., by image processing unit 112 of system 100 of FIG. 1) of one or more images of the surgical cavity that capture visual events associated with a maximum allowable pressure (e.g., a maximum allowable pressure that avoids tissue damage) being reached. For example, one or more images may be analyzed to determine that the joint space is getting excessively large over time (indicating that tissue distention is occurring) and/or that the rate of fluid passing through surgical site (such as based on tissue waving) is excessive.

In one or more examples, a machine learning model (e.g., executed by image processing unit 112) can be trained to identify such visual events (e.g., in real-time) and provide notification to the user to reduce pressure and/or send instruction to the pump to reduce pressure. A high-fidelity cadaveric simulator can be created to generate a training dataset with ground-truth labels for training the machine learning model. In this simulated environment, surgical videos can be recorded showing the use of various instruments along with a surgical pump pumping fluid into the simulated surgical cavity. The internal pressure of the simulated surgical cavity can be modulated and such modulated pressure can be used for labels for each video fragment. For example, labels could include a binary outcome (whether or not an adverse event happened, e.g., excessive tissue distension, tissue rupture, etc.), along with the maximum pressure recorded for that video fragment. The training dataset can be used to train a suitable machine learning model or combination of machine learning models.

An exemplary machine learning model is described in Pangal et al., Expert Surgeons and Deep Learning Models Can Predict the Outcome of Surgical Hemorrhage from 1 Min of Video. Sci Rep. 2022 May 17; 12(1):8137. doi: 10.1038/s41598-022-11549-2. PMID: 35581213; PMCID: PMC9114003, which is hereby incorporated by reference in its entirety. This model includes two types of networks: a convolutional neural network (CNN) for spatial feature encoding and a long short-term memory network (LSTM) that models the temporal component of an input. The CNN (e.g., a ResNet model) is initialized with the weights pre-trained on a classification dataset (ImageNet) and then fine-tuned on a surgical dataset. Alternatively, unsupervised pre-training on surgical videos and/or different architectures for the feature extraction component (e.g., transformers) can be used to train the CNN. Once trained, the machine learning model can accept a sequence of video frames as an input and predict a current pressure within the surgical site, along with the probability of an adverse event resulting from excessive pressure. When the predicted probability exceeds a pre-defined threshold, the system (e.g., image processing unit 112) can take an action, such as provide (or trigger) a warning or alarm (visual and/or audio). The system may automatically enter a mode ion which it can accept voice commands and/or confirmations from the surgeon associated with reducing pressure. Additionally or alternatively, the system can automatically reduce the pressure by controlling the pump and/or the suction (e.g., provided that the system detects one or both in the surgical site) and/or generate a control command to do so.

In one or more examples, if the process 1500 at step 1506 determines that a maximum pressure in the surgical cavity has been reached (either because any higher pressure would cause damage or because the surgical pump cannot achieve a higher pressure), then the process 1500 can move to step 1508 wherein the surgeon is notified that the surgical pump is at maximum pressure. In one or more examples, the notification may take the form of a visual display or audible tone that is configured to alert the surgeon that the image is not clear but that the pressure cannot be increased.

In one or more examples, if the process 1500 determines at step 1506 that the surgical pump is not at max pressure, then the process 1500 at step 1506 can move to step 1510 wherein the pressure, flow, and/or suction provided by the surgical pump is quickly increased (or a control command thereto is generated) in an attempt to clear or minimize visual disturbances in the surgical cavity. In one or more examples of the disclosure, the pressure exerted by the surgical pump can be increased using a proportional-integral-derivative (PID) algorithm so as to increase the pressure in a controlled and accurate manner. In one or more examples, the pressure exerted by the surgical pump can be increased using a Predictive Function Control (PFC) to control the increase or decrease in the pressure applied by the surgical pump.

Referring back to step 1504, in one or more examples, if it is determined that the image is clear, then the process 1500 can move to step 1512 wherein a determination is made as to whether the surgical pump is at its minimum allowable pressure setting. As described above, the goal of the surgical pump can be to apply the least amount of pressure to a surgical cavity as possible to minimize the risk of damage or injury to the patient. Thus, in one or more examples, in addition to increasing pressure to remove visual disturbances, the process 1500 can be configured to decrease the pressure in the joint if it is determined that there are no visual disturbances and the image is clear. A determination that the image is clear can present an opportunity for the surgical pump to reduce the pressure (because it may not be needed). Thus, at step 1512, if it is determined that the device is already at the minimum pressure needed, then the process 1500 can move to step 1514 wherein the surgical pump is not adjusted. This pressure level can be context dependent. For instance, the minimum allowable pressure for a knee surgery may be different from the minimum allowable pressure for a shoulder surgery. If, however, a determination is made that the surgical pump is not at its minimum setting at step 1512, then the process 1500 can move to step 1516 wherein the pressure exerted by the surgical pump can be reduced, which can be done, for example, by reducing a head pressure, reducing a flow rate to the surgical cavity, and/or increasing a suction in the surgical cavity. In one or more examples of the disclosure, the pressure exerted by the surgical pump can be decreased using a PID algorithm so as to decrease the pressure in a controlled and accurate manner.

In one or more examples, the settings of a surgical pump can be based, at least in part, on which of a plurality of different modes the surgical pump is operating. Different modes may prioritize different aspects of the fluid flow generated in the surgical cavity. For example, a mode may be configured to prioritize visibility in the surgical cavity and when in this mode the surgical pump can maintain a relatively high pressure and/or flow rate in the surgical cavity. Conversely, a mode may be configured to prioritize reducing the pressure over the course of a surgical procedure, which can be important in minimizing the risk of extravasation caused by excessive pressure in the surgical cavity, and as such, the surgical pump when in this mode may seek to keep the pressure in the surgical cavity relatively low over the course of the surgical procedure.

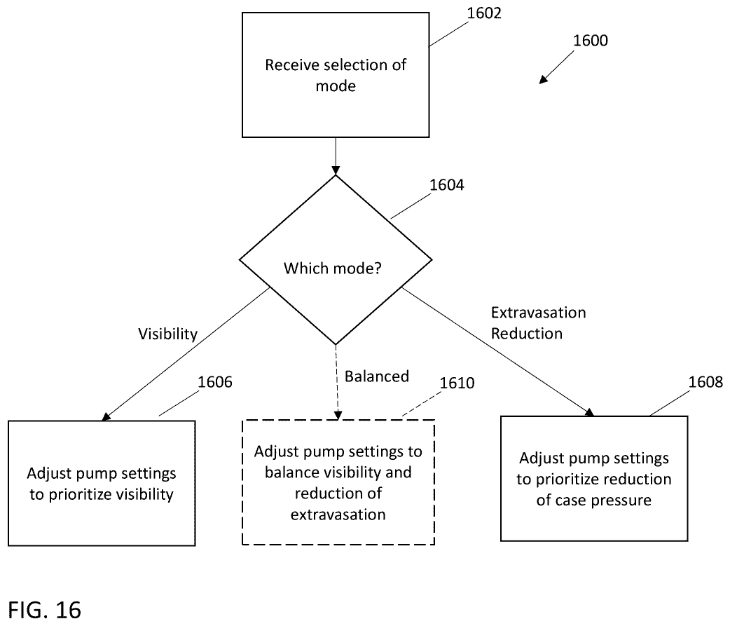

FIG. 16 is a block diagram of an exemplary process 1600 for adjusting surgical pump settings based on a selection of a mode of operation of the surgical pump. At step 1602, a selection of a mode of operation of the surgical pump is received by the surgical pump. In one or more examples, the selection can be received from a user, such as via a user input to the surgical pump and/or to a system communicatively coupled to the surgical pump. In one or more examples, the selection can be received at the surgical pump from a computing system that manages presets, such as presets associated with predefined user preferences (e.g., predefined surgeon preferences), presets associated with surgery types, and/or presets associated with patient attributes (e.g., age, sex, weight, etc.).

At step 1604, a determination is made by the surgical pump which mode has been selected. In the illustrated example, the mode can be a visibility mode that prioritizes visibility in the surgical cavity or can be a pressure reduction mode that prioritizes reducing pressure over the course of the surgical procedure (e.g., minimization of the average pressure over the course of the surgical procedure), which may minimize the risk of excessive fluid pressure causing extravasation. If the selected mode is the visibility mode, then at step 1606, the surgical pump may generate a control command to adjust, and/or adjust, its settings to prioritize visibility in the surgical cavity, such as by increasing the flow rate, increasing the head pressure, increasing the suction in the surgical cavity, and/or pulsing pressure in the surgical cavity. Optionally, in the visualization mode, higher pressures can be used for longer durations (e.g., relative to the pressure reduction mode) to keep the field of view clear. Optionally, in the visibility mode, suction can be increased to higher percentages of maximum suction capability to remove debris, blood, and/or bubbles faster, which may be coupled with monitoring for joint collapse (e.g., monitoring for a determination from joint collapse module 310 of FIG. 3 that the joint is collapsed) to ensure that the joint is not collapsing (and respond with reduced suction if it is), as discussed above.

If the selected mode is the pressure reduction mode, then at step 1608, the surgical pump can generate a control command to adjust, and/or adjust, its settings to prioritize reducing the pressure in the surgical cavity over the course of the surgical procedure. This can include adjusting the settings to reduce the pressure in the surgical cavity, such as by reducing a head pressure of the surgical pump, reducing a flow rate to the surgical cavity, and/or increasing suction provided by one or more instruments in the surgical cavity.

In one or more examples, the surgical pump can be operated in an optional balanced mode that seeks to balance the priorities of the visibility and pressure reduction modes. Thus, process 1600 may optionally include selection of a balanced mode in step 1602, which leads to step 1610, in which the surgical pump settings are adjusted to balance the priorities of visibility and pressure reduction. This can include keeping the pressure of the flow provided by the surgical pump in a range that is substantially or entirely below a pressure range associated with the visibility mode and substantially or entirely above a pressure range associated with the pressure reduction mode.

In one or more examples, each mode can be associated with different ranges of one or more characteristics of flow provided by the surgical pump. For example, each mode can have a different pump head pressure range, a different flow rate range, and/or a different suction range. The surgical pump can be configured to implement one or more of the processes described herein while in one or more of the different modes. For example, while in the pressure reduction mode, the surgical pump settings can be adjusted to increase pressure, inflow flow rate, and/or suction in the surgical cavity when characteristics affecting clarity are detected in the images and/or video data captured by the endoscopic imaging device, such as according to process 200, process 700, and/or process 800. However, these increases may be constrained by ranges associated with the respective mode. For example, the range of pressures that the surgical pump may use to clear debris may be lower when in the pressure reduction mode than when in the visibility mode. Additionally, time periods over which high pressures can be used may be shorter when in the pressure reduction mode than when in the visibility mode.

The surgical pump settings, according to any of the processes described above, may be adjusted using various control algorithms. For example, a PID control algorithm may be used to control pump pressure, pump flow rate, and/or pump suction to one or more instruments. Alternatively, surgical pump control can be done by a DNN controller.

In one or more examples, a DNN controller used to control the surgical pump can be generated using model-free reinforcement learning. In one or more examples, a DNN controller can be trained using a trained DNN-based simulator that generates simulated training data sets on which the DNN controller can be trained. The DNN simulator is trained using training data from a physical system, either a physical system operating in real-world scenarios (such as a surgical pump used during a surgical procedure) or a physical system used in a prototyped environment (such as a surgical pump used to control flow through a prototyped surgical cavity). The trained DNN controller can be used to control the surgical pump, such as in step 210 of process 200.

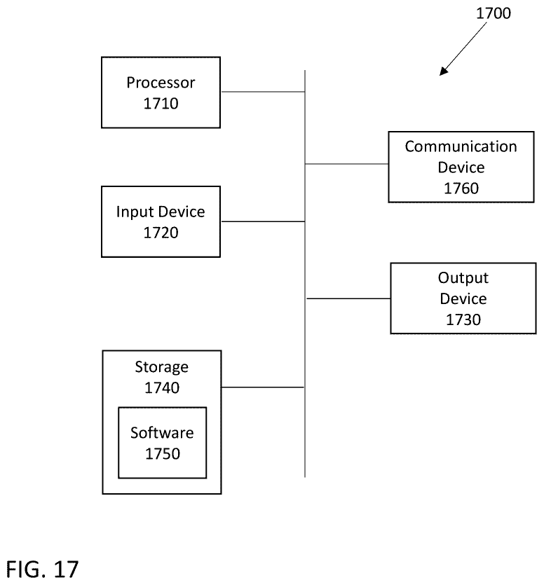

FIG. 17 illustrates an example of a computing system 1700, in accordance with some examples, that can be used for one or more components of system 100 of FIG. 1, such as one or more components of camera head 108, camera control unit 116, and image processing unit 112. System 1700 can be a computer connected to a network, such as one or more networks of hospital, including a local area network within a room of a medical facility and a network linking different portions of the medical facility. System 1700 can be a client or a server. As shown in FIG. 17, system 1700 can be any suitable type of processor-based system, such as a personal computer, workstation, server, handheld computing device (portable electronic device) such as a phone or tablet, or dedicated device. The system 1700 can include, for example, one or more of input device 1720, output device 1730, one or more processors 1710, storage 1740, and communication device 1760. Input device 1720 and output device 1730 can generally correspond to those described above and can either be connectable or integrated with the computer.

Input device 1720 can be any suitable device that provides input, such as a touch screen, keyboard or keypad, mouse, gesture recognition component of a virtual/augmented reality system, or voice-recognition device. Output device 1730 can be or include any suitable device that provides output, such as a display, touch screen, haptics device, virtual/augmented reality display, or speaker.

Storage 1740 can be any suitable device that provides storage, such as an electrical, magnetic, or optical memory including a RAM, cache, hard drive, removable storage disk, or other non-transitory computer readable medium. Communication device 1760 can include any suitable device capable of transmitting and receiving signals over a network, such as a network interface chip or device. The components of the computing system 1700 can be connected in any suitable manner, such as via a physical bus or wirelessly.

Processor(s) 1710 can be any suitable processor or combination of processors, including any of, or any combination of, a central processing unit (CPU), field programmable gate array (FPGA), application-specific integrated circuit (ASIC), and a graphical processing unit (GPU). Software 1750, which can be stored in storage 1740 and executed by one or more processors 1710, can include, for example, the programming that embodies the functionality or portions of the functionality of the present disclosure (e.g., as embodied in the devices as described above).

Software 1750 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1740, that can contain or store programming for use by or in connection with an instruction execution system, apparatus, or device.

Software 1750 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport computer readable medium can include, but is not limited to, an electronic, magnetic, optical, electromagnetic, or infrared wired or wireless propagation medium.

System 1700 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, Ti or T3 lines, cable networks, DSL, or telephone lines.

System 1700 can implement any operating system suitable for operating on the network. Software 1750 can be written in any suitable programming language, such as C, C++, Java, or Python. In various examples, application software embodying the functionality of the present disclosure can be deployed in different configurations, such as in a client/server arrangement or through a Web browser as a Web-based application or Web service, for example.

The foregoing description, for the purpose of explanation, has been described with reference to specific examples. However, the illustrative discussions above are not intended to be exhaustive or to limit the invention to the precise forms disclosed. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated. For the purpose of clarity and a concise description, features are described herein as part of the same or separate examples; however, it will be appreciated that the scope of the disclosure includes examples having combinations of all or some of the features described.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A method for coordinating the operation of a surgical instrument and a surgical pump to control fluid pressure in an internal anatomy of a patient during a surgical procedure, the method comprising:
receiving video data captured by an imaging device configured to image the internal anatomy of the patient;
automatically determining a presence of a surgical instrument in the internal anatomy of the patient based on the received video data; and
in response to determining that the surgical instrument is present in the internal anatomy of the patient, initiating a suction associated with the surgical instrument prior to activation of the surgical instrument.

2. The method of claim 1, further comprising adjusting one or more characteristics of fluid inflow to the internal anatomy generated by the surgical pump.

3. The method of claim 1, further comprising:
determining that the surgical instrument has been deactivated;
in response to determining that the surgical tool has been deactivated:
determining whether the surgical instrument is still present in the internal anatomy of the patient based on the received video data, and
in response to determining that the surgical instrument is still present in the internal anatomy of the patient, maintaining at least some of the suction associated with the surgical instrument.

4. The method of claim 3, wherein maintaining at least some of the suction comprises reducing an amount of the suction.

5. The method of claim 1, further comprising:
determining that the surgical instrument has been removed from the internal anatomy of the patient based on the received video data; and
in response to determining that the surgical instrument has been removed from the internal anatomy of the patient, deactivating the suction associated with the surgical instrument.

6. The method of claim 1, wherein automatically determining a presence of a surgical instrument in the internal anatomy of the patient based on the received video data comprises using one or more classifiers to determine the presence of the surgical instrument in the internal anatomy of the patient.

7. The method of claim 6, wherein the one or more classifiers are configured to identify at least one of a radio frequency (RF) probe, a bur tool, and a cutter tool.

8. The method of claim 1, further comprising:
determining that an instrument supplying a fluid flow to the internal anatomy of the patient has been removed from the internal anatomy of the patient by applying the one or more classifiers to the received video data; and
in response to determining that the instrument supplying the fluid flow to the internal anatomy of the patient has been removed from the internal anatomy of the patient, decreasing a flow through the surgical pump.

9. The method of claim 1, further comprising:
determining that the internal anatomy of the patient is collapsing based on the received video data; and
in response to determining that the internal anatomy of the patient is collapsing, decreasing the suction associated with the surgical instrument.

10. The method of claim 9, wherein the suction associated with the surgical instrument is decreased in response to determining that the internal anatomy of the patient is collapsing only if the surgical instrument is active.

11. The method of claim 1, further comprising:
receiving a user selection to switch a mode of operation of the surgical pump; and
in response to receiving the user selection, switching the mode of operation by adjusting at least one of: (i) the suction associated with the surgical instrument and (ii) one or more characteristics of fluid inflow to the internal anatomy generated by the surgical pump,
wherein when switching the mode comprises switching to a visualization mode, the at least one of the suction associated with the surgical instrument and the one or more characteristics of fluid inflow to the internal anatomy generated by the surgical pump is adjusted to increase at least one of a pressure and a rate of flow of fluid in the internal anatomy of the patient,
wherein when switching the mode comprises switching to a pressure reduction mode, the at least one of the suction associated with the surgical instrument and the one or more characteristics of fluid inflow to the internal anatomy generated by the surgical pump is adjusted to decrease the fluid pressure in the internal anatomy of the patient,
wherein when switching the mode comprises switching from a visualization mode or a pressure reduction mode to a balanced mode, at least one of the suction associated with the surgical instrument and the one or more characteristics of fluid inflow to the internal anatomy is adjusted to control the fluid pressure in the internal anatomy of the patient to be lower than a fluid pressure associated with the visualization mode and higher than a fluid pressure associated with the extravasation mode,
wherein the method further comprises:
receiving blood pressure information from a blood pressure monitoring device; and
adjusting at least one of: (i) the suction associated with the surgical instrument and (ii) one or more characteristics of fluid inflow to the internal anatomy generated by the surgical pump based on the received blood pressure information to increase a pressure in the internal anatomy above a mean arterial pressure.

12. The method of claim 11, comprising automatically determining a presence of blood in the internal anatomy of the patient based on the received video data, and in response to determining the presence of the blood in the internal anatomy of the patient, adjusting the at least one of the suction associated with the surgical instrument and the one or more characteristics of fluid inflow to the internal anatomy generated by the surgical pump based on the received blood pressure information.

13. A system comprising one or more processors, memory, and one or more programs stored in the memory for execution by the one or more processors and including instructions for:
receiving video data captured by an imaging device configured to image the internal anatomy of the patient;
automatically determining a presence of a surgical instrument in the internal anatomy of the patient based on the received video data; and
in response to determining that the surgical instrument is present in the internal anatomy of the patient, initiating a suction associated with the surgical instrument prior to activation of the surgical instrument.

14. The system of claim 13, wherein the one or more programs include instructions for adjusting one or more characteristics of fluid inflow to the internal anatomy generated by the surgical pump.

15. The system of claim 13, wherein the one or more programs include instructions for:
determining that the surgical instrument has been deactivated;
in response to determining that the surgical tool has been deactivated:
determining whether the surgical instrument is still present in the internal anatomy of the patient based on the received video data, and
in response to determining that the surgical instrument is still present in the internal anatomy of the patient, maintaining at least some of the suction associated with the surgical instrument.

16. The system of claim 13, wherein the one or more programs include instructions for:
determining that the surgical instrument has been removed from the internal anatomy of the patient based on the received video data; and
in response to determining that the surgical instrument has been removed from the internal anatomy of the patient, deactivating the suction associated with the surgical instrument.

17. The system of claim 13, wherein automatically determining a presence of a surgical instrument in the internal anatomy of the patient based on the received video data comprises using one or more classifiers to determine the presence of the surgical instrument in the internal anatomy of the patient, wherein the one or more classifiers are configured to identify at least one of a radio frequency (RF) probe, a bur tool, and a cutter tool.

18. The system of claim 13, wherein the one or more programs include instructions for:
determining that an instrument supplying a fluid flow to the internal anatomy of the patient has been removed from the internal anatomy of the patient by applying the one or more classifiers to the received video data; and
in response to determining that the instrument supplying the fluid flow to the internal anatomy of the patient has been removed from the internal anatomy of the patient, decreasing a flow through the surgical pump.

19. The system of claim 13, wherein the one or more programs include instructions for:
receiving a user selection to switch a mode of operation of the surgical pump; and
in response to receiving the user selection, switching the mode of operation by adjusting at least one of: (i) the suction associated with the surgical instrument and (ii) one or more characteristics of fluid inflow to the internal anatomy generated by the surgical pump.

20. A non-transitory computer readable storage medium storing one or more programs for execution by one or more processors of a computing system for causing the computing system to perform a method comprising:
receiving video data captured by an imaging device configured to image the internal anatomy of the patient;
automatically determining a presence of a surgical instrument in the internal anatomy of the patient based on the received video data; and
in response to determining that the surgical instrument is present in the internal anatomy of the patient, initiating a suction associated with the surgical instrument prior to activation of the surgical instrument.
